(19) United States
(12) Patent Application Publication
(10) Pub. No.: US 2010/0254965 A1
(43) Pub. Date: Oct. 7, 2010

(54) Adenosine Signaling in Diagnosis, Treatment, and Prevention of Priapism and Erectile Dysfunction

(75) Inventors: Yang Xia, Bellaire, TX (US); Rodney E. Kellems, Ballaire, TX (US); Michael Blackburn, Pearland, TX (US); Janci Chunn Lindsay, Sealy, TX (US)

Correspondence Address: WILLIAMS, MORGAN & AMERSON, 10333 RICHMOND, SUITE 1100, HOUSTON, TX, 77042, US

(73) Assignee: BOARD OF REGENTS ,THE UNIVERSITY OF TEXAS SYSTEM

(21) Appl. No.: 12/063,589

(22) PCT Filed: Oct. 24, 2007
(86) PCT No.: PCT/US2007/022520

§ 371 Date: May 26, 2010

Related U.S. Application Data
(60) Provisional application No. 60/862,637, filed on Oct. 24, 2006.

(51) Int. Cl. A61K 38/46; A61K 31/7028; A61K 31/522; A61K 31/52; A61K 31/472; A61P 15/10

(52) U.S. Cl. 424/94.6; 514/25; 514/263.34; 514/263.23; 514/307

(57) ABSTRACT

We disclose a method of treating priapism in a mammal by administering to the mammal a composition containing an effective amount of an inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal. We also disclose a method of treating erectile dysfunction in a mammal by administering to the mammal a composition containing an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal. In addition, we disclose a method of diagnosing erectile dysfunction in a mammal by administering to the mammal a composition containing an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal, and observing blood flow in the mammal's penis.

The United States government owns rights in the present invention pursuant to grant numbers R21 DK077748, RO1 DK046207, and RO1 HL070952 from the National Institutes of Health.

### BACKGROUND OF THE INVENTION

The present invention relates generally to the treatment of priapism or erectile dysfunction. More particularly, it concerns the treatment of priapism or erectile dysfunction by the administration of compounds that inhibit or activate adenosine signaling, respectively.

The tone of vascular smooth muscle cells is an important regulator of penile erection and is determined by the balance of vascular smooth muscle relaxants and constrictors in the penis³. An imbalance of vascular regulators may lead to either erectile dysfunction, caused by overconstriction of vascular smooth muscle, or priapism, a condition of persistent penile erection in the absence of sexual arousal, caused by overrelaxation of vascular smooth muscle. Priapism can quickly lead to anoxia, an increase in pCO₂, and acidosis, leading to erectile tissue damage and eventual erectile dysfunction¹¹⁻¹⁴. One population especially susceptible to priapism are patients with sickle cell disease (SCD): about 30% of males under the age of 20 who suffer from SCD, and about 30-45% of older males who suffer from SCD, have reported at least one priapic episode⁷⁻¹⁰.

To date, the bulk of research regarding molecular mechanisms of erectile dysfunction and priapism has focused on nitric oxide (NO)^{4,20}. NO is well-known to be a potent vasodilator, and the physiology of erection is known to involve NO release by the corpus cavernosum of the penis, where it activates guanylyl cyclase, generating cyclic guanylate monophosphate (cGMP), which leads through various intermediate steps to intracavernosal vasodilation and subsequent erection. Sildenafil (Viagra), tadalafil (Cialis), and vardenafil (Levitra) act by inhibiting the activity of cGMP-specific phosphodiesterase type 5 (PDE5), thereby allowing higher cGMP levels to arise and leading to greater intracavernosal vasodilation.

However, sildenafil and other known compounds which act in the signaling pathway downstream from guanylyl cyclase are known to have a number of side effects, including potential vision impairment, hypotension, infarction, and stroke. Also, by increasing intracavernosal vasodilation, they promote erection and are therefore not suitable for treatment of priapism. In fact, priapism is a known side effect of these drugs.

Therefore, a need exists for alternative treatments for erectile dysfunction.

Known treatments of priapism generally involve surgical intervention, frequently starting with aspiration of blood from the corpus cavernosum, and in more extreme cases progressing to shunting various routes of circulation in the penis. As can be readily imagined, these treatments can be painful and mentally discomforting. Further, aspiration or shunting does not address potential underlying causes of the priapic episode.

Therefore, a need exists for treatments of priapism that are relatively non-invasive and, if possible, capable of addressing underlying causes of priapism.

### SUMMARY OF THE INVENTION

In one embodiment, the present invention relates to a method of treating priapism in a mammal by administering to the mammal a composition containing an effective amount of an inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal.

In one embodiment, the present invention relates to a method of treating erectile dysfunction in a mammal by administering to the mammal a composition containing an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal.

In one embodiment, the present invention relates to a method of diagnosing erectile dysfunction in a mammal by administering to the mammal a composition containing an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal, and observing blood flow in the mammal's penis.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows our current understanding of the roles of NO and adenosine in normal and abnormal penile erection.

FIG. 2. Wild type mouse contains the highest level of adenosine and the lowest ADA activity in the penis, relative to other organs, and ADA-deficient mice exhibit a marked increase in adenosine concentration, particularly in penis. (A) HPLC measurement shows that the penis contains the highest level of adenosine in both wild type and ADA-deficient mice, and that the level in ADA-deficient mouse penis is roughly 4-fold higher than in wild type mouse penis. (B) ADA activity is the lowest in the penis among all the tissues measured in wild type mouse. The high level of adenosine and low ADA activity in wild type mouse penis suggests that local high level of adenosine plays a role on physiological penile vascular regulation. Because of the low levels of ADA enzyme level in penis, high levels of adenosine can last for an extended period of time and may contribute to the maintenance of penile erection. The data are displayed as mean±SE. n=5.

FIG. 3. ADA-deficient mice display spontaneous prolonged penile erection, lasting from 8-72 hr, similar to the appearance of priapism in man, associated with elevated adenosine and correctable by PEG-ADA injection (not shown).

FIG. 4. ADA-deficient mice are hypersensitive to cavernous nerve stimulation, relative to wild type mice.

FIG. 5. Administration of adenosine induced an increase in intracavernous pressure (ICP) and its duration in wild type mice. Inhibition of ADA activity by coinjection with deoxycoformycin (DCF) prolonged the adenosine-induced increase in pressure.

FIG. 6. Different amounts of adenosine were injected into the corpus cavernosum of wild type, adenosine receptor A_{2A}-deficient, and adenosine receptor A_{2B}-deficient mice and the resulting change in ICP determined. The results show that the adenosine-induced increase in ICP was significantly reduced in A_{2B}-deficient mice.

FIG. 7. Fibrotic marker gene expression in the penes of ADA-deficient mice was detected by quantitative PCR and compared with wild-type control. Data are presented as mean±SE. *, p<0.001.

FIG. 8 shows expression levels, as determined by quantitative RT-PCR, for all four adenosine receptors in mouse penis. Transcript levels of β-actin are used as a control.

FIG. 9 shows expression levels, as determined by quantitative RT-PCR, for all four adenosine receptors in mouse penile vascular smooth muscle cells (PVSM). Transcript levels of β-actin are used as a control.

FIG. 10 shows adenosine induces cAMP via A_{2B} receptor in primary mouse penile vascular smooth muscle cells (PVSM). PVSM were isolated and cultured from mouse penes. The cells were be switched to serum-free medium and treated with different concentrations of adenosine with or without A_{2B} antagonist (MRS1706-1 μM) or A_{2A} agonist (CGS-21680-1 μM) alone for 10 min. Then cellular extracts were collected and cAMP levels were measured using an ELISA kit. The data represent mean±SE.

FIG. 11 shows cAMP production in wild-type and A_{2B}^{-/-} mouse PVSM in negative controls and in the presence of forskolin (an activator of adenylyl cyclase, positive control), adenosine, and 5′-N-ethylcarbosamide adenosine (NECA). Data are presented as mean±SE. *, p<0.01.

FIG. 12 shows cGMP production, an indicator of NO signaling, in mouse penile culture as a function of adenosine concentration in wild type mouse, wild type mouse cotreated with an A_{2B} receptor antagonist, wild type mouse cotreated with nitro-L-arginine methyl ester (L-NAME, an inhibitor of nitric oxide synthase), and A_{2B}^{-/-} mouse.

FIG. 13 shows cGMP production in wild-type and A_{2B}^{-/-} mouse PVSM in negative controls and in the presence of forskolin (an activator of adenylyl cyclase, positive control), adenosine, 5′-N-ethylcarbosamide adenosine (NECA), and diethylamine-NONOate (DEA/NO), a nitric oxide donor. Results for wild-type and A_{2B}^{-/-} mouse were statistically significant, p<0.01.

FIG. 14. ADA deficient mice display spontaneous prolonged penile erection associated with increased corpus cavernosum relaxation in response to nerve stimulation. (a) ADA-deficient mice (ADA−/−) exhibited prolonged penile erection, lasting from 8-72 hours, similar to the feature of priapism in human. However, priapic erections were never observed in ADA+ male control mice. (b and c) Increased corpus cavernosum strips (CCS) relaxation by electrical field stimulation (EFS) in ADA deficient mice. Relaxation effect of EFS with different voltages (0-30V) at 30 Hz and 0.5 ms pulse width, alternating polarity between pulses (b) and frequency (0-30 Hz) at 20V and 0.5 ms pulse width, alternating polarity between pulses (c) in 10 μM phenylephrine-precontracted CCS from ADA+ (n=8) and ADA−/− mice (n=10).

FIG. 15. Priapic activity seen in ADA-deficient mice is dependent on elevated adenosine in penis. (a) Adenosine levels in the penis increased in ADA-deficient mice. Perchloric acid extracts of penile tissues from ADA-deficient mice and control mice were analyzed by HPLC to determine the levels of adenosine. Blue (ADA−/−), Red (ADA+). The inserted bar graph shows the average adenosine levels from three ADA deficient mice and three wild type mice. The data are expressed as mean±SE. *p<0.005, (ADA+ n=5 and ADA−/− n=7), ADA−/−versus the control ADA+. (b) Prolonged penile erection seen in ADA-deficient mice is due to high levels of adenosine in the penis. The prolonged penile erection in ADA-deficient mice was corrected by intraperitoneal injection of PEG-ADA, an enzyme therapy procedure reduces the circulating levels of adenosine. N=3-5. (c) Representative recording of relaxation by EFS (5V and 30 Hz) in 10 μM phenylephrine (PE)-precontracted corpus cavernosum strips (CCS) of ADA+ mice (upper) and ADA−/− mice (lower) treated with or without PEG-ADA. (d-f) Average EFS-induced relaxation from the PE-precontracted CC strips of ADA+, ADA−/− mice and ADA−/− mice treated with PEG-ADA. EFS-induced force change (d), duration of relaxation by EFS (e) and area under basal line (f) were representative of the CCS relaxation. (n=5 for ADA+ and n=6 for −/− with or without PEG-ADA treatment, respectively). Data are given as means±SE. *p<0.05, ADA−/− versus the control ADA+.

FIG. 16. (a) Inhibition of ADA activity by DCF (deoxycoformycin) increased the adenosine induced relaxation of CCS in wild type mice. CCS of wild type mice were treated with different concentrations of adenosine with or without DCF (5 μM), a specific inhibitor of adenosine deaminase. Then the extent (mN) of force was monitored. (b) CCS of wild type mice were treated with 100 μM adenosine in the presence of different concentrations of DCF (0-100 uM). Then the extent of force was measured. Data are expressed as mean±SE. *p<0.05, adenosine treated versus the control, **p<0.05, DCF treated versus the adenosine treated.

FIG. 17. A2BR signaling is required for adenosine-mediated corpus cavernosal strips (CCS) relaxation. (a) CCS of wild type mice were treated with different concentrations of theophiline (1-10 μM) in the presence or absence of adenosine (100 μM). The force changes were measured by force transducer. Data are expressed as mean±SE. *p<0.05, adenosine treated versus the control, **p<0.05, theophiline treated versus the untreated, N=5. (b) CCS from wild type, A2AR, A1R A2BR and A3R-deficient mice were treated with different concentrations of adenosine. The relaxation was measured by force transducer. Data are expressed as mean±SE. *p<0.05, adenosine treated versus the wild type. (c) CCS were isolated from wild type and A2BR-deficient mice and treated with different concentrations of adenosine in the presence or absence of L-NAME (100 μM) or MRS1706 (10 μM). 10 min after treatment, cellular extracts were collected and cAMP levels were measured by using a commercial enzyme immunoassay kit (Amersham Pharmacia). Data are expressed as mean±SE. *p<0.05, adenosine treated versus the control without treatment; **p<0.05, adenosine treated with MRS1706 versus the adenosine treated alone; ***p<0.05, CCS of A2BR-deficient mice treated with adenosine versus the CCS of wild type mice treated with adenosine. N=6-7. (d) CCS of A2BR and ADA double deficient mice lost the hypersensitivity to nerve stimulation compared to ADA deficient mice. In addition, A2BR deficient mice are less sensitive to nerve stimulation, suggesting that A2BR is also important for normal penile relaxation.

FIG. 18. Adenosine stimulated both cAMP and cGMP induction in corpus cavernosal smooth muscle cells (CCSM) via A2BR activation. (a) Adenosine receptor expression profile in purified primary CCSM. CCSM were purified from adult mouse penes. Total RNA were collected from cultured primary CCSM and quantitative RT-PCR results show that A 2B is the predominant adenosine receptor expressed in the cells. (b) CCSM were isolated and cultured from mouse penes. The cells were switched to serum-free medium and treated with different concentrations of adenosine with or without A2BR antagonist (MRS1706-10 μM), A2AR antagonist (ZM241385-50 nM) or A2AR agonist (CGS-21680-10 μM) alone for 10 min. Then cellular extracts were collected and cAMP levels were measured using an ELISA kit. The data represent mean±SE. *p<0.05, adenosine treated versus the untreated; **p<0.05, adenosine plus MRS1706 or ZM241385 versus the adenosine treated. (c) CCSM were isolated from penes of the wild type and A2BR-deficient mice and cultured with or without adenosine (100 μM), NECA (10 μM), a potent adenosine receptor agonist, forskolin (10 μM), an adenylyl cyclase agonist. After 10 min treatment, the cellular extracts were collected and cAMP levels were measured by using a commercial enzyme immunoassay kit (Amersham Pharmacia). N=4. *p<0.05, adenosine, forskolin and NECA treated versus the untreated control. (d) Corpus cavernosal strips (CCS) were isolated from wild type and A2BR-deficient mice and treated with different concentrations of adenosine in the presence or absence of L-NAME (100 μM) or MRS1706 (10 μM). 10 min after treatment, cellular extracts were collected and cGMP levels were measured by using a commercial enzyme immunoassay kit (Amersham Pharmacia). Data are expressed as mean±SE. *p<0.05, adenosine treated versus the control; **p<0.05, adenosine treated with MRS1706 versus the adenosine treated; ***p<0.05, CCS of A2BR-deficient mice treated with adenosine versus the CCS of wild type mice treated with adenosine. N=6-7. (e) CCSM were isolated from wild type and A2BR deficient mice and cultured with or without adenosine (100 μM), NECA (10 μM), a potent adenosine receptor agonist, forskolin (10 μM), an adenylyl cyclase agonist, or diethylamine-NONOate (DEA/NO) (10 μM). After 10 min treatment, the cellular extracts were collected and cAMP levels were measured by using a commercial enzyme immunoassay kit (Amersham Pharmacia). N=4. *p<0.05, DEA/NO treated versus to the untreated control. (f) Both cAMP and cGMP induction contribute to adenosine-mediated decreased PE-induced phosphorylation of myosin light chain (MLC). CCSM and CCS were isolated and cultured with the treatment of phenylephrine (PE-10 μM) in the presence or absence of adenosine (100 μM) or L-NAME (100 μM). 10 min after treatment, cellular extracts were isolated and western blot analysis were performed. The total MLC and phosphorylated MLC at S20 were detected by specific antibodies.

FIG. 19. EFS leads to increased cAMP and cGMP production via A2BR in ADA-deficient mice. (a-b) Corpus cavernosal strips (CCS) of the ADA−/− and ADA+ mice were isolated and treated with 10 μM phenylephrine (PE) to induce precontraction. Then CCS were treated with MRS1706 (10 nM), ZM241385 (50 nM) or L-NAME (100 μM) prior to electrical field stimulation (EFS) at 5V and 30 Hz. 60 second after nerve stimulation, the CCS were quickly frozen and cellular cAMP (a) and cGMP (b) were measured by using commercial enzyme immunoassay kit (Amersham Pharmacia). N=4. Data are expressed as mean±SE. *p<0.05, ADA deficient mice versus the control; **p<0.05, CCS of ADA-deficient mice with MRS1706 and/or L-NAME treatment versus CCS of ADA-deficient mice without treatment; ***p<0.05, CCS of the control mice with MRS1706 and/or L-NAME treatment versus CCS of the control mice without treatment.

FIG. 20. ADA deficient mice develop penile vascular damage and fibrosis subsequent to priapism. (a-d) Histological examination of the vascular structures in the corpus spongiosum. a-b: H&E staining; c-d: anti-smooth muscle α-actin (SMA) immunohistochemical staining. (100× magnification) (e-h) Fibrosis in corpus cavernosum and corpus spongiosum by Masson trichrome staining. (200× magnification). e-f: corpus sponsgiosum; g-h: corpus cavernosum. (i) Fibrotic marker gene expression is induced in penes of ADA deficient mice. Quantitative-PCR was performed to detect changes in the expression of profibrotic genes in penes of wild type and ADA-deficient mice. Data are presented as mean±SE. *, p<0.005, ADA−/− versus the control ADA+.

FIG. 21. Priapic activity seen in SCD transgenic mice is dependent on elevated adenosine in penis via A2BR signaling. (a) Adenosine levels in the penis increased in SCD transgenic mice. Perchloric acid extracts of penile tissues from SCD transgenic mice and control mice were analyzed by HPLC to determine the levels of adenosine. The data are expressed as mean±SE. *p<0.005, (control n=5 and SCD transgenic mice n=6), SCD transgenic mice versus the controls. (b) Increased corpus cavernosum strips (CCS) relaxation by electrical field stimulation (EFS) in SCD transgenic mice. Relaxation effect of EFS with different voltages (0-30V) at 30 Hz and 0.5 ms pulse width, alternating polarity between pulses, in 10 μM phenylephrine-precontracted CCS from the controls (n=5) and SCD transgenic mice (n=5). (c-e) Prolonged penile erection seen in SCD transgenic mice is due to high levels of adenosine in the penis via A2BR signaling. Average EFS-induced relaxation from the PE-precontracted CC strips of the controls and SCD transgenic mice treated without or with PEG-ADA, MRS1706 or ZM241389. EFS-induced force change (c), duration of relaxation by EFS (d) and area under basal line (e) were representative of the CCS relaxation. (n=5 for the controls and n=5 for SCD transgenic mice with or without PEG-ADA, MRS1706 or ZM241389 treatment, respectively). Data are given as means±SE. *p<0.05, SCD transgenic mice versus the controls, **p<0.05, CCS of ADA-deficient mice with PEG-ADA, MRS1706 or ZM241389 treatment versus CCS of SCD transgenic mice without treatment; ***p<0.05, CCS of the control mice with MRS1706 and/or L-NAME treatment versus CCS of the control mice without treatment.

### DESCRIPTION OF ILLUSTRATIVE EMBODIMENTS

Adenosine shares multiple features with NO which merit its study in penile erection. 1) Both are well known potent vasodilators and neurotransmitters. 2) Both have a very short half life. 3) Both induce cyclic nucleotide second messengers and penile erection. Specifically, adenosine functions through G protein coupled receptors (A₁, A_{2A}, A_{2B}, and A₃) to modulate adenylyl cyclase and the synthesis of cAMP11-14. Particularly, A₁ and A₃ adenosine receptors are coupled to adenylyl cyclase by the inhibitory G-protein (Gαi) and hence serve to lower intracellular levels of the second messenger cAMP. The A_{2A} and A_{2B} adenosine receptors are commonly coupled to adenylyl cyclase by the stimulatory G-protein (Gas) and serve to increase intracellular cAMP. NO functions by activating guanylyl cyclase to stimulate the synthesis of cGMP. 4) Adenosine mediated cAMP induction and NO-mediated cGMP induction are capable of inducing protein kinase A and protein kinase G, respectively, resulting in decreased calcium-calmodulin dependent myosin light chain phosphorylation and cavernosal smooth muscle relaxation, respectively²⁹. Our findings regarding the spontaneous prolonged penile erection and potent vascular relaxation in ADA deficient mice is the first genetic evidence that elevated adenosine contribute to priapism, overly relaxed penile vascular tone. In addition, our unexpected findings that flaccid wild type penis stores the highest levels of adenosine with the lowest levels of ADA activity are the novel findings implying that adenosine may function as both neurotransmitter and vasodilator to regulate penile vascular tone under physiological condition. This view is supported by earlier studies in multiple animal models, including humans, showing that intracavernous injection of adenosine resulted in tumescence and penile erection²⁹⁻³⁵. Theophyline, an adenosine receptor antagonist, inhibited adenosine-induced penile tumescence²³. These findings support our hypothesis that adenosine signaling contributes to normal penile vascular regulation via receptor activation.

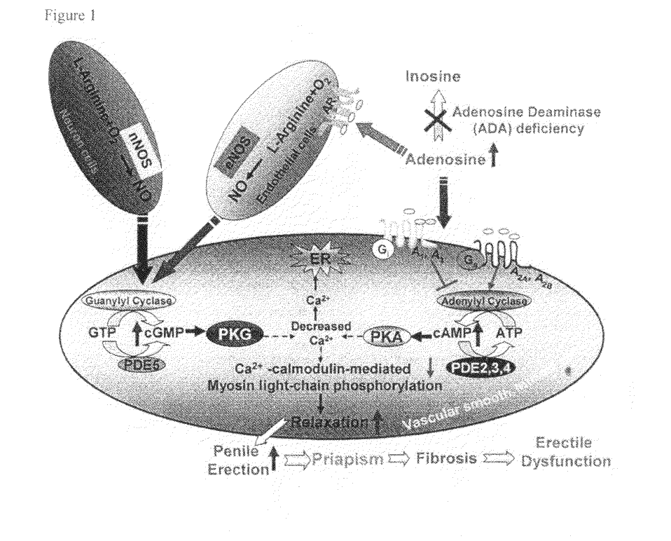

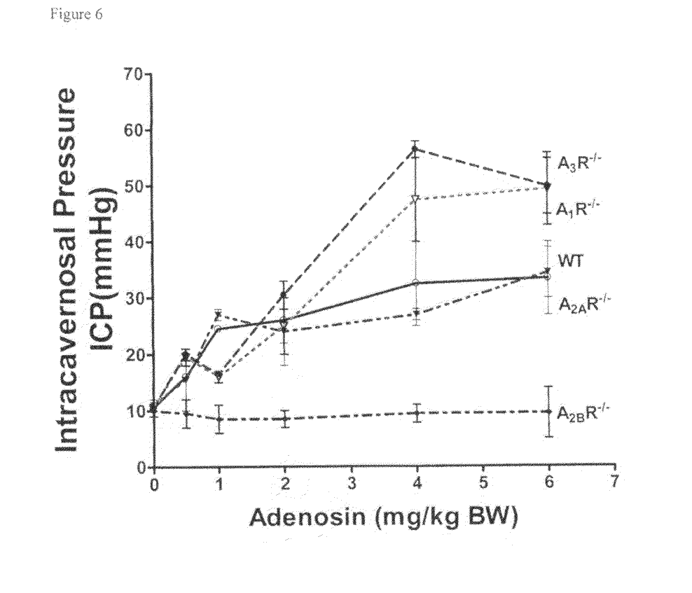

Adenosine is a signaling nucleoside that elicits its effect on target cells by engaging specific G-protein coupled receptors³⁷ (FIG. 1). Four such receptors have been described, A₁, A_{2A}, A_{2B}, and A₃. Each receptor has a unique affinity for adenosine and a distinct cellular and tissue distribution. Most evidence suggests that the A₁ and A₃ adenosine receptors are coupled to adenylyl cyclase by the inhibitory G-protein (Gαi) and hence serve to lower intracellular levels of the second messenger cAMP³⁸⁻⁴¹. The A_{2A} and A_{2B} adenosine receptors are commonly coupled to adenylyl cyclase by the stimulatory G-protein (Gas) and serve to increase intracellular cAMP^{41,42}. Evidence also exists to suggest that these receptors couple to other effector molecules such as phospholipase C and PI3 kinase^{40,42}. Therefore, signaling through adenosine receptors may play important roles in the regulation of both intracellular cAMP and Ca²⁺ and can hence influence cellular physiology in a number of ways. Adenosine signaling plays important roles in regulating homeostasis in a number of physiological systems including the cardiovascular⁴³, nervous⁴⁴, renal⁴⁵, and immune systems^{46,47}. On one hand, adenosine protects tissues like the brain^{48,49} and heart⁵⁰ from ischemic damage and exhibits chemoprotective properties⁵¹. On another hand, there are situations in which adenosine signaling functions to promote or exacerbate tissue injury and fibrosis. Such are the cases with adenosine-mediated mast cell degranulation⁵²⁻⁵⁶ and fibroblast cell transformation from myofibroblast cells^{25,26} that are thought to influence the tissue injury and physiological alterations seen in allergic diseases such as asthma and chronic obstructive pulmonary disease. The cell type specific expression of adenosine receptors and the effector system they couple to, together with the concentration of adenosine produced in the local environment, likely dictate the beneficial or harmful impacts of this nucleoside. Using A_{2A} or A_{2B} receptor deficient mice we have preliminary data showing that deletion of A_{2B} receptor but not A_{2A} receptor inhibits adenosine induced intracavernous pressure (FIG. 6). Thus, these data suggest that A_{2B} is the major receptor involved in regulation of penile erection in response to adenosine.

FIG. 1 presents our current best understanding of the roles of NO and adenosine in normal and abnormal penile erection. Multiple cell types are involved in penile erection, including endothelial, neuronal and vascular smooth muscle cells (VSMC). NO is produced from both endothelial cells and neurons to induce cGMP production in vascular smooth muscle cells, resulting in dilation and penile erection. Similarly, adenosine induces penile erection by vascular smooth muscle relaxation through the cAMP pathway. Adenosine functions through G protein coupled adenosine receptors, i.e. A₁, A_{2A}, A_{2B}, and A₃. A₁ and A₃ are Gi coupled to inhibit cAMP synthesis, whereas A_{2A} and A_{2B} are Gs coupled to enhance cAMP production. cAMP and cGMP lead to activation of PKA and PKG, respectively. Activated PKA and PKG stimulate the uptake of Ca²⁺ into the endoplasmic reticulum (ER) resulting in vascular smooth muscle cell relaxation. We have observed prolonged penile erection and fibrosis in ADA-deficient mice, suggesting that uncontrolled accumulation of adenosine in the penes of these mice may be responsible for the priapism and penile fibrosis we observed. The research discussed in the examples below utilizes ADA-deficient mice as investigative tools to study the role of adenosine signaling in priapism.

In one embodiment, the present invention relates to a method of treating priapism in a mammal by administering to the mammal a composition containing an effective amount of an inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal.

Any mammal can be the subject of this or any other method disclosed herein. In one embodiment, the mammal is Homo sapiens. In a further embodiment, the H. sapiens has sickle cell disease or priapism resulting from use of an erectile dysfunction medication. As is known, priapism is a common side effect of erectile dysfunction medications, such as sildenafil (Viagra), tadalafil (Cialis), and vardenafil (Levitra).

In another embodiment, the mammal is a male of a non-human mammalian species. In a further embodiment, the mammal is selected from the group consisting of stallions, bulls, rams, dogs, cats, rabbits, rats, and mice.

By “treating” a disease is meant improving the condition of a subject suffering or at risk of suffering from the disease. Treating can comprise one or more of the following: a reduction in the severity of a symptom of the disease, a reduction in the extent of a symptom of the disease, a reduction in the number of symptoms of the disease, a reduction in the spread of a symptom of the disease, a delay in the onset of a symptom of the disease, a delay in disease onset, or a reduction in the time between onset of the disease and remission of the disease, among others apparent to the skilled artisan having the benefit of the present disclosure. To the extent that the foregoing examples of treating a disease are defined in relative terms, the proper comparison is to the disease or symptoms thereof when no composition is administered to treat it and no method is performed to treat it. Treating can be performed in response to the presentation of a symptom of the disease or it can be performed prophylactically, before symptoms of the disease present. Treating can be performed in response to acute presentations of a symptom of the disease or according to a schedule for treatment of a chronic disease.

An “effective amount” of an active ingredient of a composition, such as an inhibitor of adenosine signaling or, in other embodiments of the present invention, an activator of adenosine signaling, is an amount sufficient to treat the disease.

As is known to the skilled artisan, a salt or ester of an active ingredient of a composition, such as an inhibitor of adenosine signaling or, in other embodiments of the present invention, an activator of adenosine signaling, may possess comparable activity to the active ingredient in its non-salt, non-ester form, or may have another beneficial property, such as enhanced stability or reduced prevalence or severity of side effects, relative to the active ingredient. The term “inhibitor” or “activator,” when used herein, includes such salts or esters.

By “pharmaceutically-acceptable” is meant that the carrier is suitable for use in medicaments intended for administration to a mammal. Parameters which may considered to determine the pharmaceutical acceptability of a carrier can include, but are not limited to, the toxicity of the carrier, the interaction between the inhibitor of adenosine signaling or, in other embodiments of the present invention, the activator of adenosine signaling and the carrier, the approval by a regulatory body of the carrier for use in medicaments, or two or more of the foregoing, among others.

The pharmaceutically-acceptable carrier can be any material or plurality of materials which can form a composition with the inhibitor of adenosine signaling or, in other embodiments of the present invention, the activator of adenosine signaling. The particular pharmaceutically-acceptable carrier can be selected by the skilled artisan in view of the intended use of the composition and the properties of the inhibitor of adenosine signaling or, in other embodiments of the present invention, the activator of adenosine signaling, among other parameters apparent in light of the present disclosure.

Non-limiting examples of particular pharmaceutically-acceptable carriers and particular compositions follow.

In one embodiment, the pharmaceutically-acceptable carrier is water, and the composition is an aqueous solution comprising water and the inhibitor of adenosine signaling or, in other embodiments of the present invention, the activator of adenosine signaling. The composition can further comprise solutes, such as salts, acids, bases, or mixtures thereof, among others. The composition can also comprise a surfactant, an emulsifier, or another compound capable of improving the solubility of the inhibitor of adenosine signaling or, in other embodiments of the present invention, the activator of adenosine signaling in water.

In one embodiment, the pharmaceutically-acceptable carrier is a polar organic solvent, and the composition is a polar organic solution comprising the polar organic solvent and the inhibitor of adenosine signaling or, in other embodiments of the present invention, the activator of adenosine signaling. “Polar” has its standard meaning in the chemical arts of describing a molecule that has a permanent electric dipole. A polar molecule can but need not have one or more positive, negative, or both charges. Examples of polar organic solvents include, but are not limited to, methanol, ethanol, formate, acrylate, or mixtures thereof, among others. The composition can further comprise solutes, such as salts, among others. The composition can also comprise a surfactant, an emulsifier, or another compound capable of improving the solubility of the inhibitor of adenosine signaling or, in other embodiments of the present invention, the activator of adenosine signaling in the polar organic solvent.

In one embodiment, the pharmaceutically-acceptable carrier is an apolar organic solvent, and the composition is an apolar organic solution comprising the apolar organic solvent and the inhibitor of adenosine signaling or, in other embodiments of the present invention, the activator of adenosine signaling. “Apolar” has its standard meaning in the chemical arts of describing a molecule that does not have a permanent electric dipole. Examples of apolar organic solvents include, but are not limited to, hexane, cyclohexane, octane, toluene, benzene, or mixtures thereof, among others. The composition can further comprise solutes, such as apolar molecules, among others. The composition can also comprise a surfactant, an emulsifier, or another compound capable of improving the solubility of the inhibitor of adenosine signaling or, in other embodiments of the present invention, the activator of adenosine signaling in the apolar organic solvent. In one embodiment, the composition is a water-in-oil emulsion, wherein the inhibitor of adenosine signaling or, in other embodiments of the present invention, the activator of adenosine signaling is dissolved in water and water is emulsified into a continuous phase comprising one or more apolar organic solvents.

In one embodiment, the pharmaceutically-acceptable carrier is a mixture of water and other solvents. In one embodiment, the pharmaceutically-acceptable carrier can comprise one or more of dimethicone, water, urea, mineral oil, sodium lactate, polyglyceryl-3 diisostearate, ceresin, glycerin, octyldodecanol, polyglyceryl-2-dipolyhydroxystearate, isopropyl stearate, panthenol, magnesium sulfate, bisabolol, lactic acid, lanolin alcohol, or benzyl alcohol, among others.

In one embodiment, the composition has a creamy, lotion-like, or ointment-like consistency suitable for topical administration. Exemplary carriers for this embodiment include (a) dimethyl sulfoxide, optionally with a polyalcohol, a dispersant, and water, such as described in U.S. Pat. No. 4,575,515, which is hereby incorporated herein by reference; (b) lamellar aggregates comprising phospholipids, and optionally further comprising fluorocarbons, in an excipient suitable for topical administration, such as described in U.S. Pat. No. 5,686,102, which is hereby incorporated herein by reference; (c) foams, gels, creams, ointments, transdermal patches, or pastes, as described in U.S. Pat. No. 7,265,091, which is hereby incorporated herein by reference; and (d) nanoparticles, among others.

In one embodiment, the pharmaceutically-acceptable carrier is a solid or semisolid carrier, and the composition is a solid or semisolid matrix in or over which the inhibitor of adenosine signaling or, in other embodiments of the present invention, the activator of adenosine signaling is dispersed. Examples of components of solid carriers include, but are not limited to, sucrose, gelatin, gum arabic, lactose, methylcellulose, cellulose, starch, magnesium stearate, talc, petroleum jelly, or mixtures thereof, among others. The dispersal of the inhibitor of adenosine signaling or, in other embodiments of the present invention, the activator of adenosine signaling can be homogeneous (i.e., the distribution of the inhibitor of adenosine signaling or, in other embodiments of the present invention, the activator of adenosine signaling can be invariant across all regions of the composition) or heterogeneous (i.e., the distribution of the inhibitor of adenosine signaling or, in other embodiments of the present invention, the activator of adenosine signaling can vary at different regions of the composition). The composition can further comprise other materials, such as flavorants, preservatives, or stabilizers, among others.

In one embodiment, the pharmaceutically-acceptable carrier is a gas, and the composition can be a gaseous suspension of the inhibitor of adenosine signaling or, in other embodiments of the present invention, the activator of adenosine signaling in the gas, either at ambient pressure or non-ambient pressure. Examples of the gas include, but are not limited to, air, oxygen, nitrogen, or mixtures thereof, among others.

Other carriers will be apparent to the skilled artisan having the benefit of the present disclosure.

In addition to the inhibitor of adenosine signaling or, in other embodiments of the present invention, the activator of adenosine signaling and the carrier, and further components described above, the composition can also further comprise other compounds, such as preservatives, adjuvants, excipients, binders, other agents capable of treating one or more diseases, or mixtures thereof, among others. In one embodiment, the other compounds are pharmaceutically acceptable.

The concentration of the inhibitor of adenosine signaling or, in other embodiments of the present invention, the activator of adenosine signaling in the composition can vary, depending on the pharmaceutically-acceptable carrier and other parameters apparent to the skilled artisan having the benefit of the present disclosure. The concentration of other components of the composition can also vary along the same lines.

In one embodiment, the inhibitor of adenosine signaling is selected from the group consisting of adenosine deaminase (ADA), polyethylene-glycol modified adenosine deaminase (PEG-ADA), and 5′-ectonucleotidase inhibitors, and has at least the activity of decreasing adenosine levels in the mammal. Decreasing adenosine levels can involve one or more of depleting adenosine faster than adenosine is produced by the mammal or inhibiting adenosine production by the mammal, among other routes. Decreasing is determined relative to basal levels prior to administration of the inhibitor of adenosine signaling. ADA and PEG-ADA catalyze the irreversible conversion of adenosine to inosine. PEG-ADA is adenosine deaminase modified to be more stable in vivo. A 5′-ectonucleotidase inhibitor is a compound which inhibits an enzyme which converts adenylyl monophosphate (AMP) to adenosine.

In one embodiment, 5′-ectonucleotidase inhibitor is selected from the group consisting of ARL67156, APOPCP (β-Methyl ADP), and salts and esters thereof. According to the website of the manufacturer, Tocris Bioscience, Ellisville, Mo. (http://www.tocris.com/products/ARL_{—}67156_trisodium_salt.php), the trisodium salt of ARL67156 has the structure:

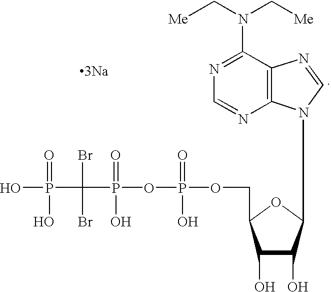

In another embodiment, the inhibitor of adenosine signaling is selected from the group consisting of theophylline, adenosine receptor A_{2B} antagonists, ZM241385, adenosine receptor A_{2A} antagonists, and salts and esters thereof, and has at least the activity of inhibiting adenosine receptor activity in the mammal. In a further embodiment, the inhibitor of adenosine signaling is selected from the group consisting of theophylline, adenosine receptor A_{2B} antagonists, adenosine receptor A_{2A} antagonists, and salts and esters thereof. Inhibiting adenosine receptor activity can involve blocking the ability of adenosine to interact with a receptor, blocking the receptor's ability to pass a signal downstream, or both, among other routes. “Inhibit” encompasses both partial and complete blocking of one or more of the abilities referred to above and is relative to basal adenosine receptor activity prior to administration of the inhibitor of adenosine signaling. Theophylline, adenosine receptor A_{2B} antagonists, ZM241385, adenosine receptor A_{2A} antagonists, and salts and esters thereof generally block the ability of adenosine to interact with a receptor.

A number of types of adenosine receptor are known, including adenosine receptor A₁, adenosine receptor A_{2A}, adenosine receptor A_{2B}, and adenosine receptor A₃. Inhibitors of adenosine receptor activity may inhibit one or more specific adenosine receptors or may generally inhibit all adenosine receptors. In a further embodiment, the inhibitor of adenosine signaling has at least the activity of inhibiting adenosine receptor A_{2B} activity in the mammal. In yet another embodiment, the inhibitor of adenosine signaling has at least the activity of inhibiting adenosine receptor A_{2A} activity in the mammal. Theophylline is a general adenosine receptor antagonist. ZM241385 is typically considered an adenosine receptor A_{2A} antagonist. Enprophylline, MRS1706, and MRS1754 are typically considered adenosine receptor A_{2B} antagonists.

In one embodiment, the adenosine receptor A_{2B} antagonist is selected from the group consisting of MRS1706, MRS1754, and CVT-6883. Both MRS1706 and MRS1754 are products of Merck & Co., Inc., Whitehouse Station, N.J., and CVT-6883 is a product of CV Therapeutics, Inc., Palo Alto, Calif. Structural and other information concerning CVT-6883 was reported by Kalla, et al., U.S. Pat. No. 6,825,349.

In another embodiment, the inhibitor of adenosine signaling is selected from the group consisting of adenylyl cyclase inhibitors, protein kinase A inhibitors, and salts and esters thereof, and has at least the activity of inhibiting signaling pathways downstream of an adenosine receptor in the mammal. Inhibiting signaling pathways downstream of an adenosine receptor can involve blocking the ability of the adenosine receptor to pass a signal to a downstream protein, enzyme, or other molecule, blocking the ability of the signal to bind to a downstream protein, enzyme, or other molecule, blocking the ability of a downstream protein, enzyme, or other molecule to pass a signal to a further downstream protein, enzyme, or other molecule, or two or more thereof, among other routes. Though not to be bound by theory, as shown in FIG. 1, at least some adenosine receptors are believed to pass a signal downstream to adenylyl cyclase, which then is believed to pass a signal to protein kinase A, which, when activated, stimulates the uptake of Ca²⁺ into the endoplasmic reticulum and leads to vascular smooth muscle cell relaxation. “Inhibit” encompasses both partial and complete blocking of one or more of the abilities referred to above and is relative to basal activity prior to administration of the inhibitor of adenosine signaling.

In one further embodiment, the inhibitor of adenosine signaling is an adenylyl cyclase inhibitor selected from the group consisting of SQ22536 and salts and esters thereof. In another further embodiment, the inhibitor of adenosine signaling is a protein kinase A inhibitor selected from the group consisting of H-89, PIK₁₄₋₂₂, and salts and esters thereof.

The signaling pathway inhibited downstream of an adenosine receptor can be downstream from any adenosine receptor. In one embodiment, the signaling pathway is downstream from an adenosine receptor selected from the group consisting of adenosine receptor A_{2B} and adenosine receptor A_{2A}.

Administration can involve any technique for introducing the composition into the mammal. Administration can be systemic, that is, the composition is not directly delivered to a tissue, tissue type, organ, or organ system the function of which is impaired by an oxidative stress disease, or it can be localized, that is, the composition is directly delivered to a tissue, tissue type, organ, or organ system the function of which is impaired by an oxidative stress disease. The route of administration can be varied, depending on the composition and the disease, among other parameters, as a matter of routine experimentation by the skilled artisan having the benefit of the present disclosure. Exemplary routes of administration include transdermal, subcutaneous, intravenous, intraarterial, intramuscular, intrathecal, intraperitoneal, oral, rectal, and nasal, among others. In one embodiment, the route of administration is oral or intravenous.

For treating priapism, localized administration of the composition to the target tissue can be desirable, both to reduce side effects in non-target tissues and to potentially allow smaller doses of the inhibitor of adenosine signaling. Exemplary routes of localized administration include, but are not limited to, intracavernosal, intraurethral, and transdermal. Intracavernosal injection may bring about pain and mental discomfort for the mammal, and thus may not be preferred for self administration by a human patient. A transdermal application, such as in a lotion, cream, or ointment, may be more suitable for self administration by a human patient. If the composition is to be administered by a medical professional in a clinical setting, issues of patient pain and mental discomfort may be less relevant.

In one embodiment, the present invention relates to a method of treating erectile dysfunction in a mammal by administering to the mammal a composition containing an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal.

In one embodiment, the activator of adenosine signaling is selected from the group consisting of adenosine, 5′-N-ethylcarbosamide adenosine (NECA), adenosine deaminase inhibitors, adenosine transporter inhibitors, adenosine kinase inhibitors, and salts and esters thereof, and has at least the activity of increasing adenosine levels in the mammal. Increasing adenosine levels can involve one or more of accumulating adenosine (or an analog thereof active in adenosine signaling) in the mammal's penis faster than adenosine is depleted therefrom or activating adenosine production by the mammal, among other routes. Increasing is determined relative to basal levels prior to administration of the activator of adenosine signaling. Administration of adenosine and NECA will accumulate adenosine in the mammal. Adenosine deaminase inhibitors reduce the activity of ADA, leading to less adenosine being converted to inosine. Adenosine transporter inhibitors reduce the rate of export of adenosine from the mammal's penis. Adenosine kinase inhibitors reduce the rate of phosphorylation of adenosine.

In a further embodiment, the adenosine deaminase inhibitor is selected from the group consisting of 2′-deoxycoformycin and salts and esters thereof. In another further embodiment, the adenosine transporter inhibitor is selected from the group consisting of dipyridamole and salts and esters thereof. In an additional further embodiment, the adenosine kinase inhibitor is selected from the group consisting of 5′-amino-5′-deoxyadenosine (AMDA) and salts and esters thereof.

In one embodiment, the activator of adenosine signaling is selected from the group consisting of general adenosine receptor agonists, adenosine receptor A_{2B} agonists, adenosine receptor A_{2A} agonists, and salts and esters thereof, and has at least the activity of activating adenosine receptor activity in the mammal. Activating adenosine receptor activity can involve direct interaction between the activator of adenosine signaling and a receptor, enhancing the receptor's ability to pass a signal downstream, introducing an activating signal into the pathway downstream of the receptor, or two or more thereof, among other routes. Activating is relative to basal adenosine receptor activity prior to administration of the activator of adenosine signaling. A general adenosine receptor agonist will directly interact with multiple types of adenosine receptors to activate adenosine signaling, although the parameters of the interaction between the general adenosine receptor agonist and each of the multiple types of adenosine receptors may not be the same. Adenosine receptor A_{2B} agonists are typically specific for adenosine receptor A_{2B}, although they may have a weaker interaction with other types of adenosine receptors. The analogous description applies to adenosine receptor A_{2A} agonists.

In a further embodiment, the activator of adenosine signaling has at least the activity of activating adenosine receptor A_{2B} activity in the mammal. In another further embodiment, the activator of adenosine signaling has at least the activity of activating adenosine receptor A_{2A} activity in the mammal.

In one embodiment, the activator of adenosine signaling is selected from the group consisting of adenylyl cyclase activators, cyclic adenosine monophosphate (cAMP), 8-bromo-cAMP, 6-benzyl-cAMP, 8-CPT-2′-0-methyl-cAMP, and salts and esters thereof, and has at least the activity of activating signaling pathways downstream of an adenosine receptor in the mammal. Activating signaling pathways downstream of an adenosine receptor can involve enhancing the ability of the adenosine receptor to pass a signal to a downstream protein, enzyme, or other molecule, enhancing the ability of the signal to bind to a downstream protein, enzyme, or other molecule, enhancing the ability of a downstream protein, enzyme, or other molecule to pass a signal (such as cAMP) to a further downstream protein, enzyme, or other molecule, or two or more thereof, among other routes. Adenylyl cyclase activators will promote an increase in cAMP levels, as will the administration of cAMP or a modified cAMP, such as 8-bromo-cAMP, 6-benzyl-cAMP, 8-CPT-2′-O-methyl-cAMP, and salts and esters thereof.

In a further embodiment, the adenylyl cyclase activator is selected from the group consisting of forskolin and salts and esters thereof.

The activated signaling pathway can be downstream from any adenosine receptor. In one embodiment, the activated signaling pathway is downstream of an adenosine receptor selected from the group consisting of adenosine receptor A_{2B} and adenosine receptor A_{2A}.

The composition containing the activator of adenosine signaling can be administered along much the same lines as discussed above concerning administration of a composition in the treatment of priapism. Systemic applications, such as orally by tablet, solution, or suspension, may be more convenient and more readily complied with for the treatment of erectile dysfunction than localized ones, especially intracavernosal and intraurethral injections.

In one embodiment, the present invention relates to a method of diagnosing erectile dysfunction in a mammal by (i) administering to the mammal a composition containing an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal, and (ii) observing blood flow in the mammal's penis.

The composition administered in the administering step, as well as the administration step itself, can be as described above.

In one embodiment, the activator of adenosine signaling is selected from the group consisting of adenosine, 5′-N-ethylcarbosamide adenosine (NECA), adenosine deaminase inhibitors, adenosine transporter inhibitors, adenosine kinase inhibitors, and salts and esters thereof, and has at least the activity of increasing adenosine levels in the mammal. In another embodiment, the activator of adenosine signaling is selected from the group consisting of general adenosine receptor agonists, adenosine receptor A_{2B} agonists, adenosine receptor A_{2A} agonists, and salts and esters thereof, and has at least the activity of activating adenosine receptor activity in the mammal. In a different embodiment, the activator of adenosine signaling is selected from the group consisting of adenylyl cyclase activators, cyclic adenosine monophosphate (cAMP), 8-bromo-cAMP, 6-benzyl-cAMP, 8-CPT-2′-O-methyl-cAMP, and salts and esters thereof, and has at least the activity of activating signaling pathways downstream of an adenosine receptor in the mammal.

Administering the activator of adenosine signaling will tend to promote vascular smooth muscle cell relaxation, leading to observable changes in blood flow and subsequent penile erection. Systemic or localized administration of the composition can be performed.

A number of techniques of observing blood flow in vivo are known. In one embodiment, observing blood flow is performed by Doppler sonography. As will be known to the skilled artisan, sonography involves the transmission of sound waves into a mammal's body, at least partial reflection of the sound waves by various structures in the mammal's body, receipt of the reflected sound waves, and production of an image from the received reflected sound waves. Doppler sonography further involves extraction of direction and relative velocity information about a structure or substance in motion, such as blood. By observing blood flow after administration of the activator of adenosine signaling, and possibly with comparison to observed blood flow prior to administration, the operator can determine whether the mammal's blood flow is influenced by administration of the activator of adenosine signaling, and thus diagnose whether an erectile dysfunction is caused in whole or in part by disruption in signaling, such as adenosine signaling, or if it is caused in whole or in part by another mechanism, for example, vascular occlusion or a psychological condition.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### ABSTRACT OF EXAMPLES

Introduction and aims: Priapism is defined as prolonged penile erection occurring unassociated with sexual interest. 40% of male sickle cell disease patients display priapism. The disorder is dangerous and urgent given its association with erectile tissue damage and erectile dysfunction. Current strategies to manage the disorder are poor due to lack of fundamental understanding of the pathophysiology of priapism. In recent years, numerous studies have focused on the functional role of nitric oxide (NO) in normal and abnormal penile erection. Adenosine, like NO, is a potent vasodilator, neurotransmitter and has long been implicated in regulating penile tumescence. In addition, adenosine is a signaling nucleoside that elicits many physiological effects by engaging membrane receptors. However, the role of adenosine signaling in priapism is overlooked and remains unknown. Our current findings indicate that ADA-deficient mice have a pronounced priapism phenotype, and thus may serve as an animal model to investigate the role of adenosine signaling in priapism and penile fibrosis.

The elevation of adenosine in ADA-deficient mice is a direct metabolic consequence of the enzyme deficiency. However, other conditions, such as those associated with SCD, could also result in increased concentrations of adenosine in the penis. Adenosine is a signaling molecule that is generated at sites of hypoxia, tissue injury and inflammation. Consistent with this, adenosine levels are usually elevated in tissues following ischemia. The most common form of priapism is the ischemic type. The priapism seen in sickle cell disease patients is usually induced by hypoxia or ischemia and is highly associated with hemolysis. These conditions are likely to result in high local concentrations of adenosine, sufficient to induce priapic activity. Thus, to determine if increased adenosine generally applies to the pathophysiology of priapism, and is not merely a peculiarity of ADA-deficient mice, we test the importance of adenosine signaling in contributing to the priapic phenotype associated with sickle cell disease (SCD) transgenic mice, a well accepted priapism animal model.

Methods: ADA-deficient mice and SCD transgenic mice, a well accepted priapism animal model, are the only animals known in which a persistent and prolonged penile erection occurs spontaneously. The utility of ADA-deficient mice is aided significantly by the ability to use ADA enzyme therapy as convenient experimental strategy to regulate adenosine levels, and thereby control the penile abnormalities associated with ADA deficiency. We have measured intracavernosal pressure and corpus cavernosal strip (CCS) relaxation with or without PEG-ADA treatment and/or a series of adenosine receptor antagonists or agonists in wild type ADA-deficient mice, and corpus cavernosal strip (CCS) relaxation with or without PEG-ADA treatment and/or a series of adenosine receptor antagonists or agonists in SCD transgenic mice, in response to nerve stimulation. We have also measured the intracavernosal pressure and CCS relaxation with or without adenosine treatment in wild type and multiple adenosine receptor deficient mice.

Results: As a result of ADA deficiency these mice exhibit a widespread increase in adenosine, a signaling nucleoside that elicits many physiological effects by engaging membrane receptors. The priapism phenotype is associated with the accumulation of high levels of adenosine in the penes of ADA-deficient mice. Intraperitoneal injection of PEG-ADA (a modified form of ADA used for enzyme therapy) quickly reverses the priapic activity, suggesting that the erections are due to elevated levels of adenosine. In addition, ADA-deficient mice display a hypersensitive penile erection in response to cavernous nerve stimulation. Intracavernous injection of adenosine in wild type mice results in penile erection, an outcome that was more intense and more prolonged when adenosine was co-injected with an ADA inhibitor. Finally our recent findings indicate that adenosine induced priapism requires A_{2B} receptor activation, an observation with obvious therapeutic implications.

ADA deficient mice display priapic activity associated with spontaneous prolonged penile erection, hypersensitivity to nerve stimulation, vascular damage and fibrosis, all major features of priapism seen in humans. ADA enzyme therapy successfully corrected the priapic activity both in vivo and in vitro in these mice, suggesting that the priapic activity was dependent on elevated adenosine levels. In addition, local inhibition of ADA activity in corpus cavernosal strips from wild type mice resulted in a pronounced adenosine-induced relaxation, further supporting a role for endogenous adenosine in priapism. Using adenosine receptor blockers and adenosine receptor deficient mice, we determined that the A_{2B} adenosine receptor (A_{2B}R) is required for adenosine-induced penile vascular relaxation and erection. Adenosine activation of the A_{2B}R directly stimulated cAMP production and indirectly stimulated neighboring cells to release nitric oxide (NO) resulting in cGMP induction in corpus cavernosum smooth muscle cells. Finally, we showed that priapic activity in sickle cell disease transgenic mouse, a well accepted animal model of priapism, is also due to elevated adenosine via A_{2B}R signaling.

Conclusion: Our findings indicate that adenosine signaling contributes to penile vascular tone regulation and ADA-deficient mice are a valuable animal model to investigate the role of adenosine signaling in priapism and penile fibrosis.

Thus, in two independent mouse genetic models, ADA deficiency and SCD, we have shown that A_{2B} adenosine receptor signaling contributes to priapism. The former model is a general model for studying the consequences of enhanced adenosine receptor signaling and the latter is a well accepted model of priapism. It is intriguing that an unexpected priapic phenotype associated with ADA-deficient mice led us to successfully explore the unrecognized pathophysiological importance of increased adenosine signaling in priapism observed in SCD mice. Therefore, the experimental findings with ADA-deficient mice were confirmed and extended using SCD transgenic mice, a well accepted animal model of priapism. Overall, we believe our findings with ADA deficient mice and SCD mice provide strong support for the view that A_{2B} adenosine receptor signaling contributes to priapism and that this signaling pathway represents a potentially important therapeutic target for the treatment of priapism.

### Example 1

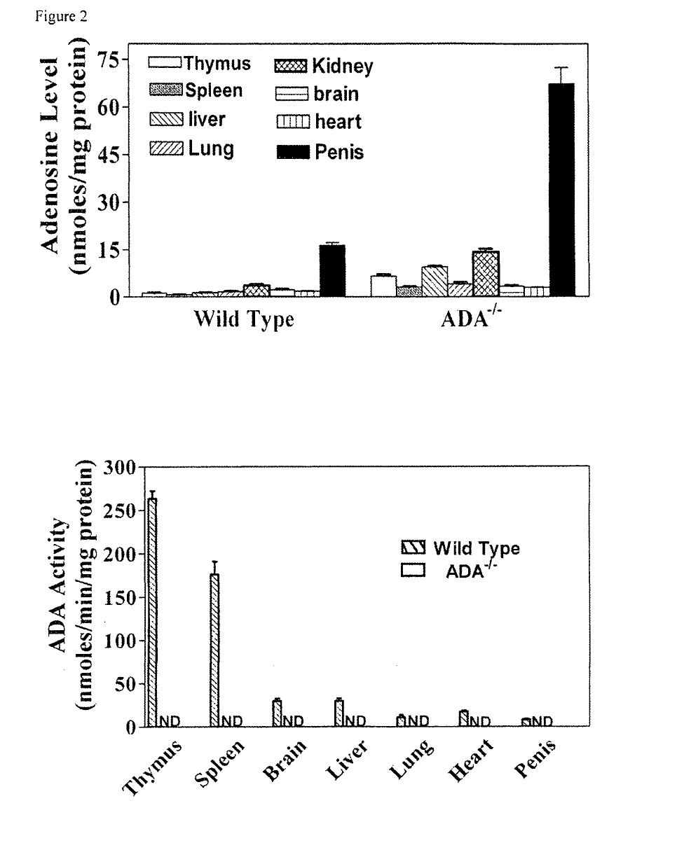

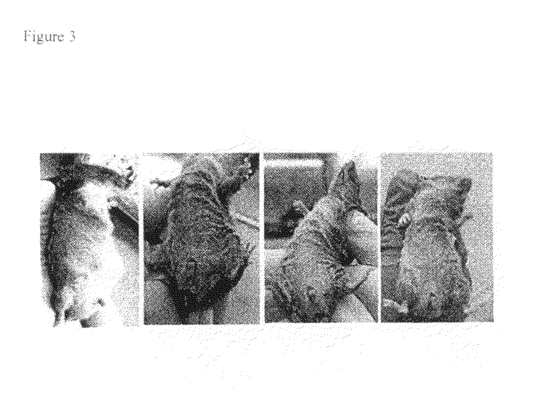

Adenosine deaminase (ADA) is a purine metabolic enzyme that catalyzes the conversion of adenosine to inosine. ADA deficient (ADA^{-/-}) mice exhibit a marked increase in adenosine concentrations²¹, particularly in the penis, which has the highest adenosine level and the lowest ADA activity among all the tissues examined in both wild type and mutant mice (FIG. 2). These results suggest that the low level of penile ADA activity in wild type mice may allow for a locally high concentration of adenosine to regulate vascular tone and thus penile erection.

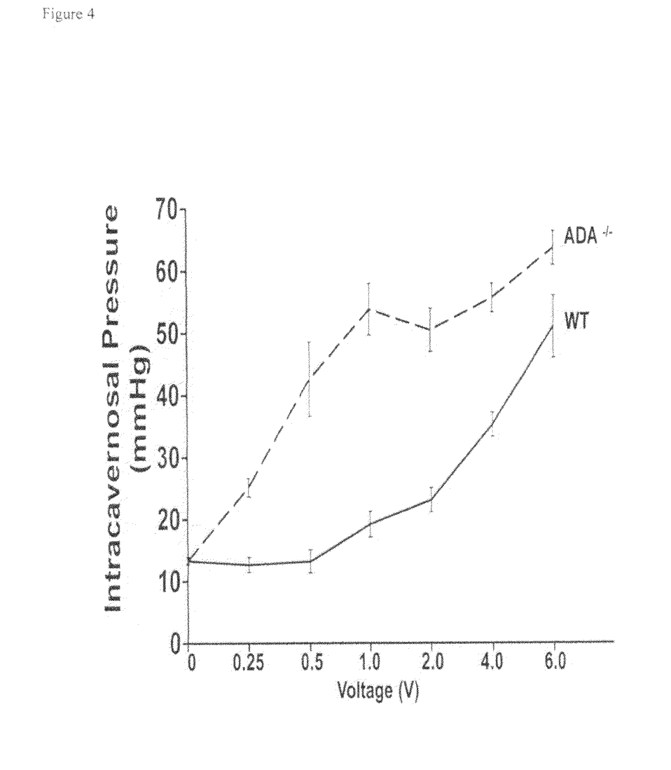

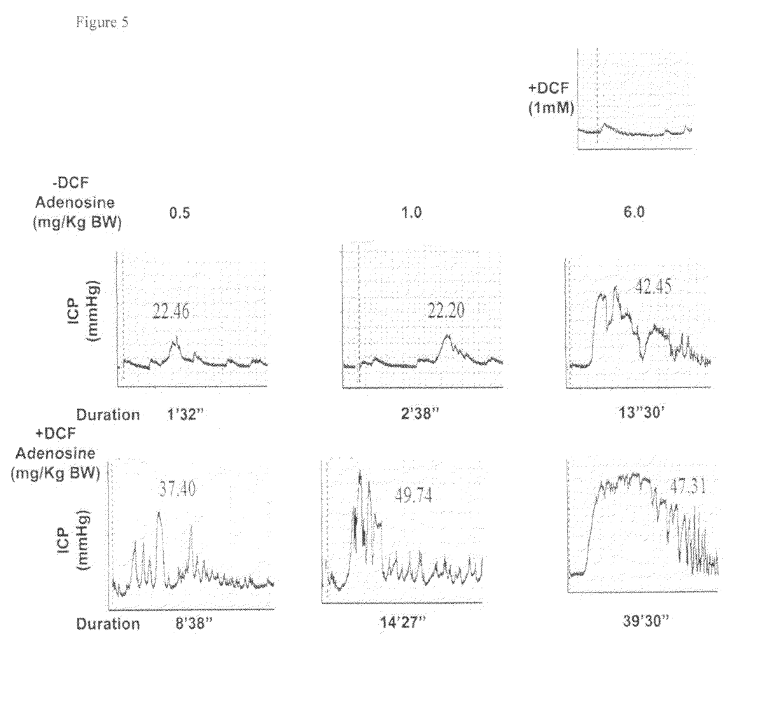

Surprisingly, ADA-deficient mice display features of priapism seen in humans, including spontaneously prolonged penile erection (FIG. 3). Intraperitoneal injection of PEG-ADA (a modified form of ADA used for enzyme therapy), relieved the prolonged penile erection. This initial finding suggests that adenosine signaling plays a major role in promoting cavernosal smooth muscle relaxation resulting in the prolonged penile erection observed in these mice. Consistent with this view, the ADA deficient mice show a hypersensitive response to cavernous nerve stimulation (FIG. 4), suggesting that accumulated adenosine in the penis may contribute to prolonged penile erection.

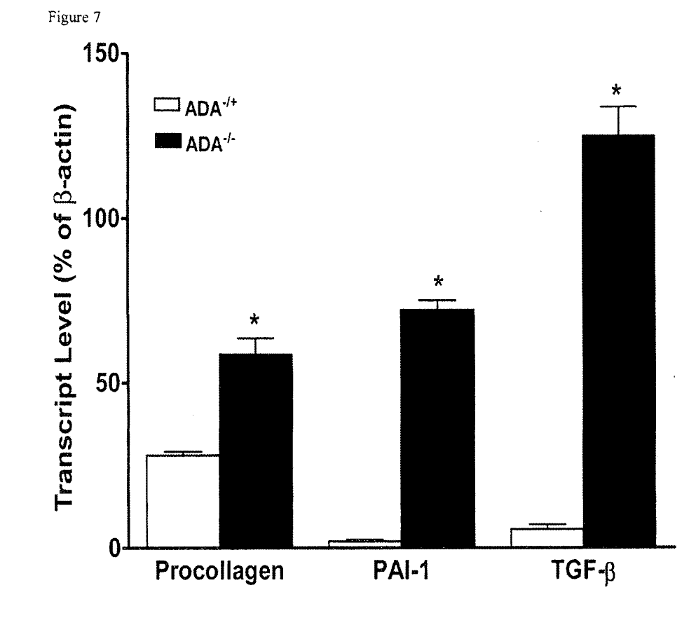

In addition, ADA-deficient mice exhibit other complications of priapism, including penile fibrosis. We used quantitative RT-PCR to monitor the expression of a series of fibrotic marker genes in ADA-deficient mice and wild type mice. FIG. 7 shows that procollagen, plasminogen activating inhibitor-1 (PAI-1), and TGF-β gene expression are significantly increased in the penes of ADA-deficient mice maintained with low dosage (1 u/wk) PEG-ADA enzyme therapy for 15 weeks. Our preliminary data suggest that ADA-deficient mice develop penile fibrosis, secondary to priapism. The chronic fibrotic changes of the penis of an ADA-deficient mouse due to priapism may eventually progress to erectile dysfunction.

Furthermore we found, in wild type mice, local injection of adenosine resulted in penile erection, an outcome that was prolonged when adenosine was co-injected with deoxycoformycin, a potent inhibitor of ADA (FIG. 5).

Consistent with a role for adenosine signaling, we found that deletion of the A_{2B} adenosine receptor in mice inhibited adenosine-induced penile erection (FIG. 6). These findings suggest that adenosine induced prolonged penile erection is through A_{2B} adenosine receptor activation, an observation with immediate therapeutic implications for priapism. Thus, we have provided extensive preliminary data to support the hypothesis that adenosine signaling plays an unexpected regulatory role in normal and abnormal penile vascular tone.

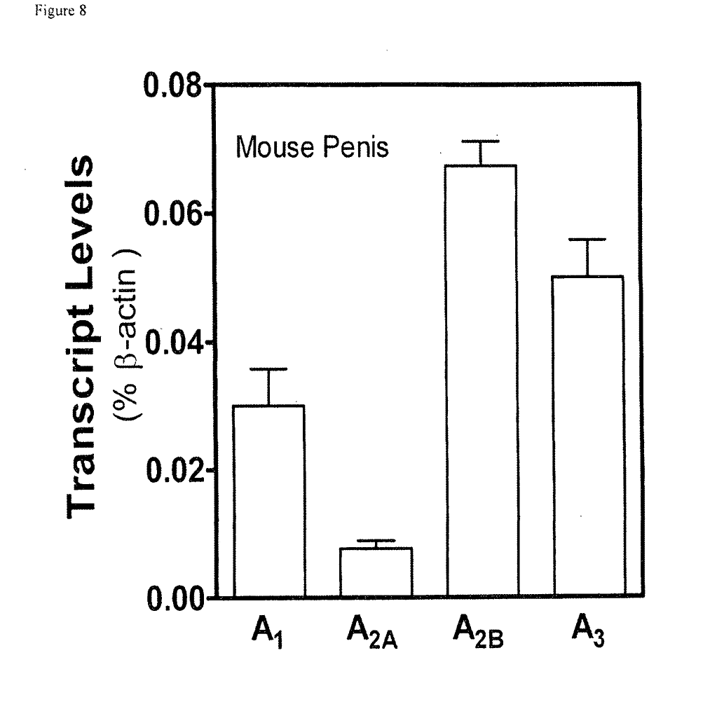

Cellular distribution of adenosine receptors and ADA in penile tissue. The principle cell types found within the penis include neuronal cells, endothelial cells, and vascular smooth muscle cells (See FIG. 1). Principle regulators of penile function are molecules produced by neuronal and endothelial cells that act on cavernosal smooth muscle (e.g., NO). Adenosine is well known as a neurotransmitter and vasodilator. We are the first to show that adenosine level in the penis of the wild type mouse is the highest among all the tissues we have measured, even including the brain (FIG. 2)^{21,24}. Thus, in the penis such a high level of adenosine may serve both functions. Our quantitative RT-PCR data (FIG. 8) indicate that all four adenosine receptors are present in the penis.

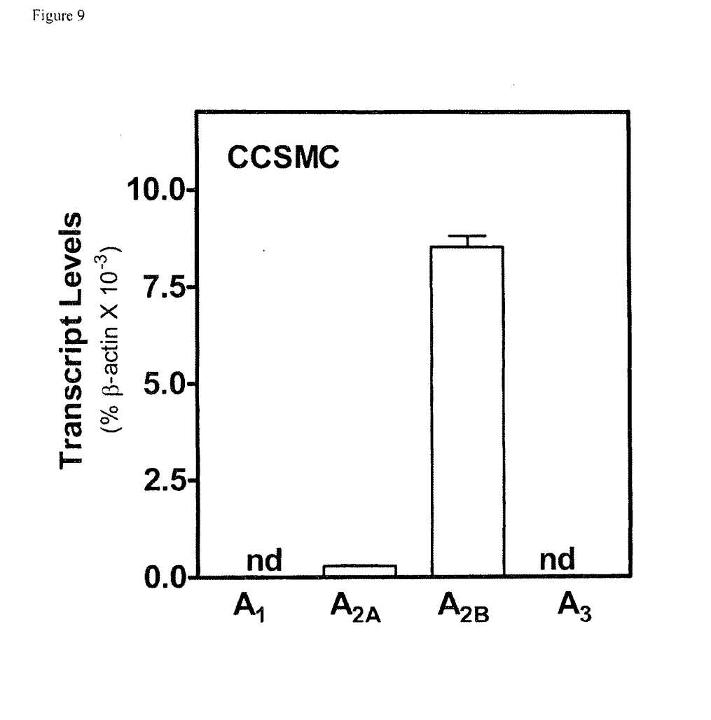

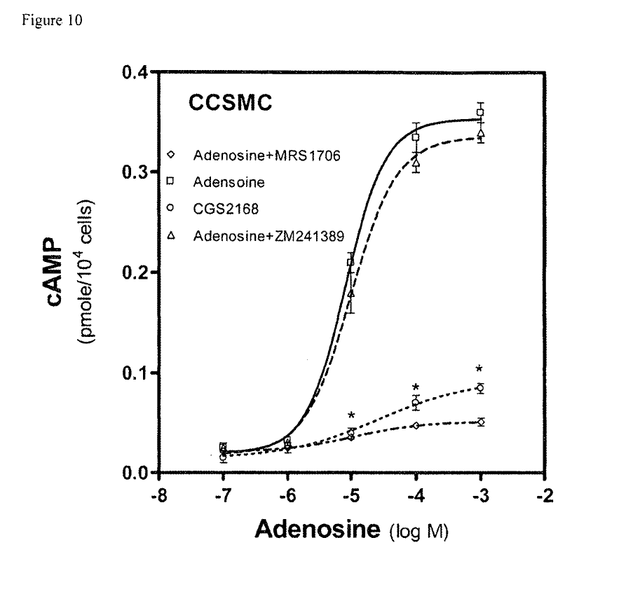

In vitro approaches to study adenosine signaling in the penis. In an effort to decipher specific cell types involved with adenosine signaling in the penis we propose to conduct experiments using primary cultures of corpus cavernosum vascular smooth muscle cells⁷⁰ or endothelial cells⁷¹. These two cell types were selected because they are likely targets of adenosine signaling. We have already successfully isolated vascular smooth muscle cells from wild type penis and used quantitative RT-PCR to determine that the predominant receptor expressed in this cell is the A_{2B} receptor (FIG. 9). Additionally, we have used adenosine receptor antagonists to show adenosine-stimulated cAMP production in these primary cultures via A_{2B} receptor activation (FIG. 10).

These results are consistent with our in vivo results showing that adenosine-induced penile erection requires the A_{2B} receptor (FIG. 6). Thus, this initial finding suggested that adenosine could act directly on cavernosum smooth muscle cells to induce cAMP production through A_{2B} adenosine receptor activation.

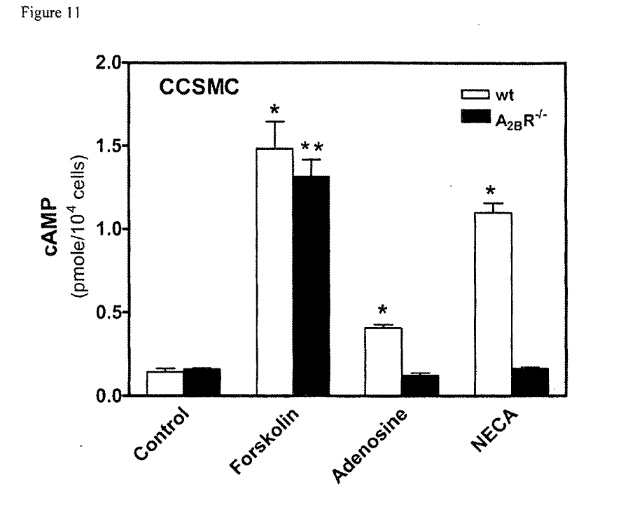

This suggestion was confirmed as shown in FIG. 11. Both wild-type and A_{2B}^{-/-} mouse PVSM produce low and comparable baseline levels of cAMP (control). Forskolin, an activator of adenylyl cyclase, allows activation of the adenosine-cAMP signaling pathway without the need for activation of A_{2B} adenosine receptor, leading to high and comparable levels of cAMP in both wild-type and A_{2B}^{-/-} mouse PVSM. Adenosine and NECA, an analog thereof, elevate cAMP levels in wild-type mouse PVSM having the A_{2B} adenosine receptor but have essentially no effect on cAMP levels in A_{2B}^{-/-} mouse PVSM. Therefore, we conclude that A_{2B} is required for adenosine mediated stimulation of cAMP production in the cavernosum smooth muscle cells. We do not expect adenosine to have any effect on smooth muscle cGMP levels.

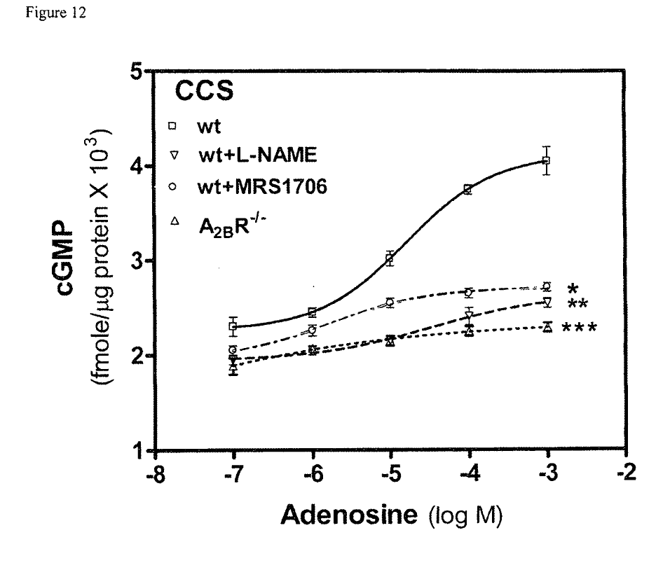

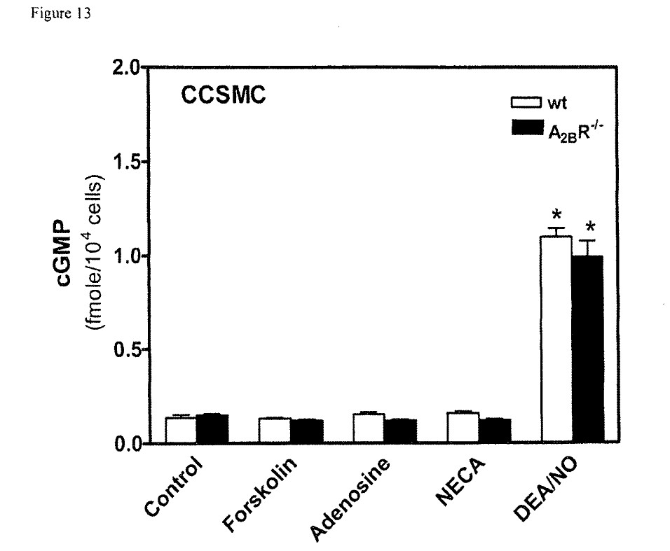

We also surmised that adenosine may also promote vascular smooth muscle relaxation by stimulating endothelial cells to produce NO that diffuses to the neighboring vascular smooth muscle cells to activate guanylyl cyclase and produce cGMP. To test this possibility possibility we performed the experiments summarized in FIGS. 12-13. Pharmacologic approaches using specific receptor agonists and antagonists were used to determine which adenosine receptor, if any, was required for the stimulation of NO production.

The functional interaction of adenosine mediated signaling events on endothelial cells and vascular smooth muscle cells was performed by the in vitro culture of cavernosal strips. Specifically, submerged cavernosal strips from wild type or A_{2B}^{-/-} mouse were incubated with different concentrations of adenosine, alone or in the presence of an A_{2B} antagonist or L-NAME (an inhibitor of nitric oxide synthase), and cGMP levels were determined. FIG. 12 indicates adenosine can increase cGMP production in penile culture via an interaction with the A_{2B} adenosine receptor. However, FIG. 13 indicates the signaling pathway from the A_{2B} adenosine receptor through adenylyl cyclase does not promote cGMP production in PVSM cells, as indicated by the lack of significant differences in cGMP production for various adenosine-adenylyl cyclase pathway activators (forskolin, adenosine, NECA) relative to the negative controls and the comparable high levels of cGMP production in both wild-type and A_{2B}^{-/-} mouse PVSM when treated with diethylamine-NONOate, a nitric oxide donor. It is likely that endothelial cells are the cell type in which adenosine-stimulated NO production occurs.

Through these studies we hope to gain insight into the intracellular mechanisms by which adenosine signaling may influence penile erection and priapism. Adenosine deaminase-deficient mice accumulate supraphysiological levels of adenosine leading to priapism. Other conditions leading to elevated adenosine (possibly sickle cell disease) may also result in priapism.

### Example 2

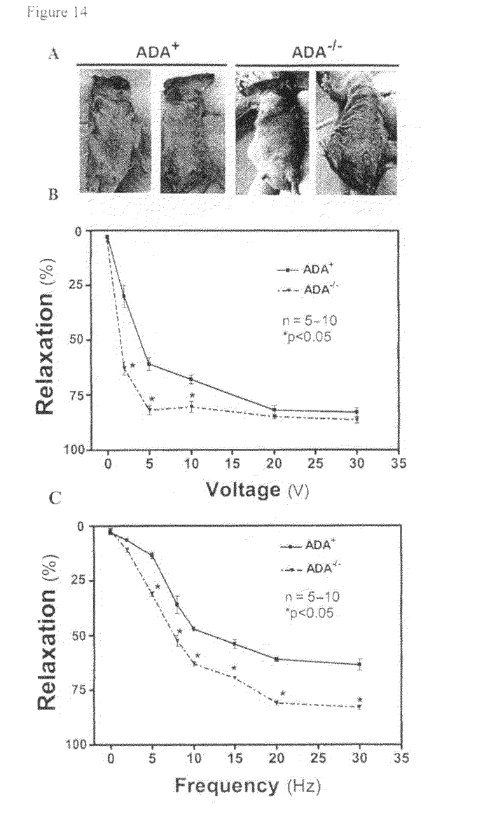

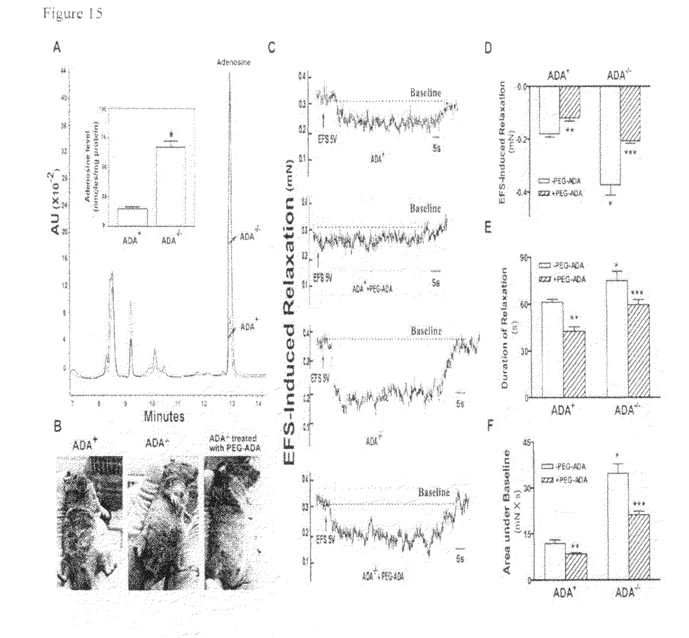

ADA-deficient mice exhibit priapic activity with spontaneous prolonged penile erection. Although ADA deficiency is a lethal condition in humans and mice, it is possible to prolong life indefinitely with the use of polyethylene glycol (PEG)-ADA enzyme therapy, a treatment that reduces (but does not eliminate) the accumulation of adenosine²⁸. The ADA-deficient mice were injected with PEG-ADA weekly. During the course of maintaining our production colony of ADA-deficient mice, we noticed that ADA-deficient male breeders (kept alive by enzyme therapy) frequently presented with prolonged penile erections lasting up to 72 hours (FIG. 1a). The penile erections were typically noticed at the time of the scheduled weekly PEG-ADA injection. We know from earlier studies that the lowest levels of circulating adenosine are achieved during the first day following the PEG-ADA injection^{21,28}. Adenosine levels rise continually during the subsequent days preceding the next PEG-ADA injection. Thus, adenosine levels are highest just prior to the scheduled weekly injection of PEG-ADA when the priapism was observed^{21,28}. Priapic erections were never observed in wild type male mice (FIG. 14a). This finding suggests that ADA-deficient mice may be a unique and valuable genetic animal model of priapism characterized by spontaneous prolonged penile erection.

Prolonged penile erection in ADA-deficient mice is associated with increased corpus cavernosal relaxation in response to nerve stimulation. To determine if the potent and prolonged penile erection seen in ADA-deficient mice is associated with increased corpus cavernosal relaxation, we evaluated comparatively the relaxation of phenylephrine-precontracted isolated corpus cavernosal strips of ADA+ and ADA−/− mice in response to nerve stimulation^{73,74}. The corpus cavernosum is under sympathetic influence to maintain a contracted state resulting in a flaccid penis³. To emulate this situation, contraction was induced by treatment with the α-adrenergic receptor agonist, phenylephrine (10 μM). The maximum phenylephrine-induced contraction was reached within 10-20 s after application to cavernosal strips from ADA+ and ADA−/− mice. Electrical field stimulation (EFS) is commonly used to stimulate the nerves that innervate the smooth muscle cells of the corpus cavernosum to mediate relaxation, corresponding to normal physiological penile erection^{19,73,74,92,93}. In our studies, EFS induced relaxation of corpus cavernosal strips from both ADA+ and ADA−/− mice in a voltage and frequency-dependent manner (FIGS. 14b and c). For ADA+ mice, relaxation increased incrementally over a standard voltage range with a maximal response at 20 V and 30 Hz. However, ADA−/− mice showed increased sensitivity to EFS and achieved maximal relaxation with 5V and 30 Hz stimulation (FIGS. 14b and c). These results suggest that the spontaneous prolonged penile erection observed in ADA-deficient mice is associated with corpus cavernosal relaxation in response to nerve stimulation.

Prolonged penile erection in ADA-deficient mice depends on pronounced accumulation of adenosine in the penis. We hypothesized that the increased penile erection and corpus cavernosal smooth muscle relaxation in response to nerve stimulation in ADA-deficient mice were due to elevated concentrations of adenosine. To test this hypothesis, adenosine levels were quantified in penile tissue from ADA⁺ and ADA^{-/-} mice^{21,23,28}. We found that ADA^{-/-} mice exhibit a marked increase in adenosine concentrations in the penis (FIG. 15a). To determine the critical role of elevated adenosine in priapic activity in ADA-deficient mice, we injected PEG-ADA into mice that presented with priapic activity. We found that the prolonged penile erections observed in ADA-deficient mice were quickly corrected by intraperitoneal injection of a high-dose of PEG-ADA (FIG. 15b). This in vivo observation provides the clue that elevated adenosine in the penes of ADA-deficient mice may be responsible for spontaneous prolonged penile erection.

Elevated adenosine in ADA-deficient mice contributes to increased corpus cavernosal strip relaxation. To further confirm our in vivo observation, we functionally tested the effect of PEG-ADA on increased relaxation in ADA-deficient mouse corporal cavernosal strips (CCS) in response to EFS as described above, which is commonly used and well accepted functional assay in multiple species^{19,73,74,92,93}. CCS of ADA-deficient mice achieved maximal relaxation with a 5V and 30 Hz stimulation (FIGS. 14b and c). Therefore, we chose to stimulate CCS of both ADA⁺ and ADA^{-/-} mice at 5V and 30 Hz for 60 s in the presence or absence of PEG-ADA treatment. Specifically, 60 s EFS at 5V and 30 Hz evoke a substantial and prolonged relaxation of CCS from ADA^{-/-} mice in comparison to CCS from ADA⁺ mice (FIG. 15c-e). A significantly greater area under baseline was observed due to the prolonged and substantial relaxation of CCS of ADA^{-/-} mice (FIG. 15f). Moreover, we found that treating CCS from ADA^{-/-} mice with PEG-ADA significantly reduced the force, duration and area under baseline when compared with untreated CCS (FIG. 15c-f). In addition, we found that PEG-ADA decreased the EFS-induced CCS relaxation in the control mice (FIG. 15c-f), suggesting that adenosine may also play a role in normal penile erection. Taken together, our functional assay with PEG-ADA treatment demonstrated that adenosine may be involved in normal physiological penile erection and elevated adenosine contributes to the prolonged and substantial penile vascular relaxation and erection associated with ADA-deficient mice.

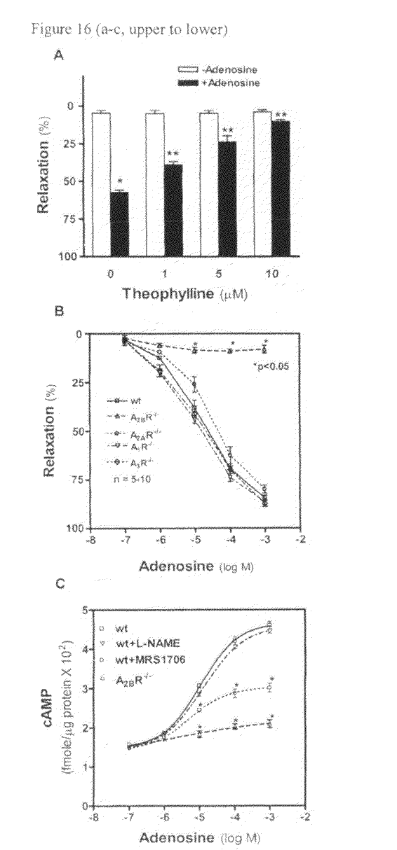

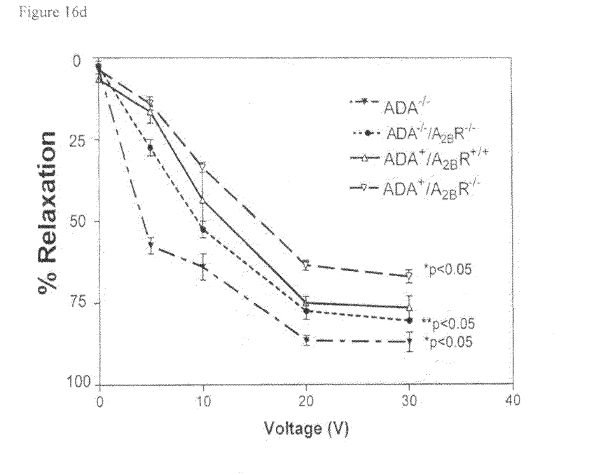

Inhibition of ADA activity in wild type mice induces potent penile corporal cavernosal strip relaxation. To directly assess the potential role of elevated adenosine in priapism, we measured the extent of relaxation in CCS from wild type mice following treatment with adenosine in the presence or absence of deoxycoformycin (DCF, a potent ADA inhibitor). We found that adenosine induced CCS relaxation in a dosage dependent manner (FIG. 16a), consistent with earlier studies in other species⁷⁷. In addition, we found that the combination of DCF plus adenosine significantly increased relaxation compared with adenosine alone (FIG. 16b). These pharmacological findings with adenosine/DCF co-incubation support our in vivo and in vitro findings that the elevated adenosine may lead to priapism.

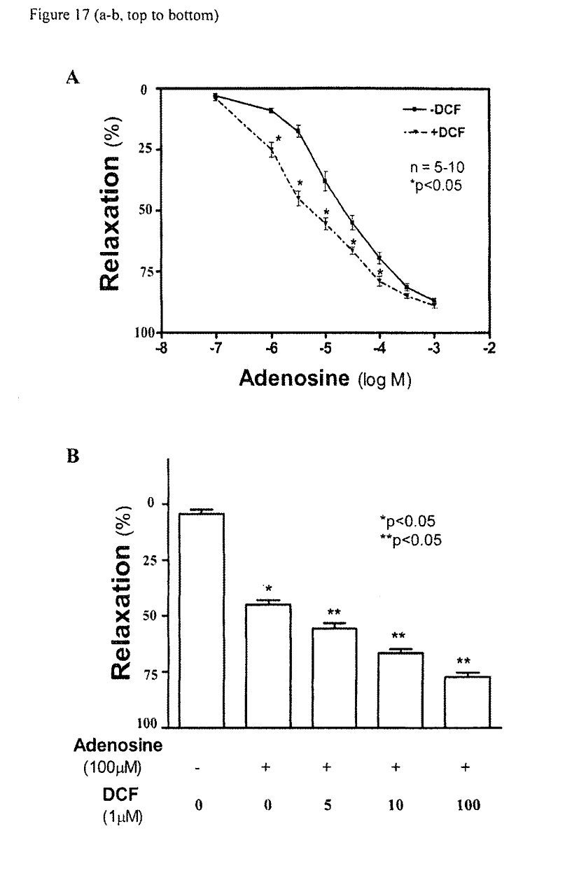

Adenosine-induced corpus cavernosal relaxation requires A₂₈ adenosine receptor. To determine whether adenosine mediated corpus cavernosal relaxation and prolonged penile erection is through adenosine receptor signaling, we measured adenosine-induced CCS relaxation in the presence of theophyline, a general adenosine receptor antagonist. We found that treatment with theophyline significantly inhibited adenosine-mediated corpus cavernosal strip relaxation in a dosage dependent manner (FIG. 17a), suggesting that adenosine-mediated corpus cavernosal relaxation is through adenosine receptor activation.

To identify which adenosine receptor is essential for adenosine-mediated penile vascular smooth muscle relaxation, we measured CCS relaxation in adenosine receptor deficient mice in response to different dosages of adenosine. We found that A₁R, A_{2A}R and A₃R deficient mice and wild type mice showed a dose-dependent increase in relaxation following treatment with adenosine. In contrast, the adenosine-mediated relaxation was completely absent in CCS from A_{2B}R deficient mice (FIG. 17b). These results provide strong genetic evidence that the A_{2B}R is required for corpus cavernosal strip relaxation in response to adenosine.

To determine the signaling components functioning downstream of the A_{2B}R, we measured the cAMP levels in response to adenosine in the isolated CCS (i.e., penile organ culture) of both wild type and A_{2B}R deficient mice. Adenosine induced cAMP levels in a dose-dependent manner in wild type but not A_{2B}R deficient penile organ culture (FIG. 17c). Similarly, we found that the adenosine-mediated induction of cAMP was inhibited by an A_{2B}R antagonist (MRS1706) but not by a NO synthase inhibitor (L-NAME) (FIG. 14c). Thus, both genetic and pharmacological studies demonstrate that adenosine acts via A_{2B}R activation to induce cAMP levels in corpus cavernosal strips and the effect is not mediated through NO signaling.

A_{2B} adenosine receptor is responsible for hypersensitivity to nerve stimulation in ADA deficient mice. To address whether A_{2B}R was responsible for increased response to nerve stimulation in ADA deficient mice, we tested CCS relaxation in response to EFS in ADA and A_{2B}R double deficient mice. As shown in FIG. 17d, CCS of A_{2B}R and ADA double deficient mice lost the hypersensitivity to nerve stimulation compared to ADA deficient mice. In addition, A_{2B}R deficient mice are less sensitive to nerve stimulation, suggesting that A2BR is also important for normal penile relaxation (FIG. 17d).

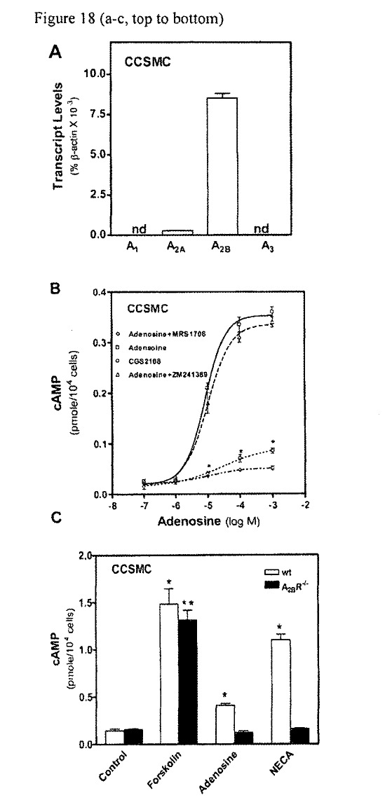

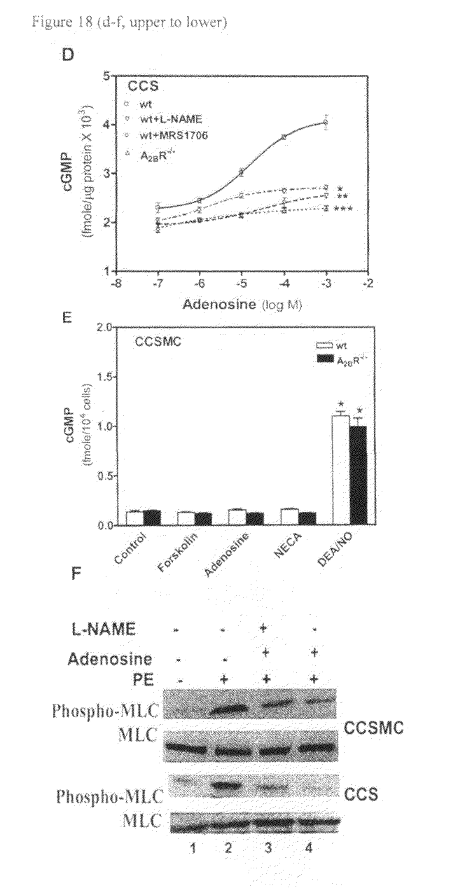

Adenosine acts directly on corpus cavernosal smooth muscle cells (CCSM) and leads to cAMP induction via A_{2B}R activation. Next, in an effort to decipher specific cell types involved with A_{2B}R signaling in the penis we purified and cultured corpus cavernosal smooth muscle cells (CCSM), the key cell type in the regulation of penile vascular tone⁷¹, from both wild type and A_{2B}R deficient mice. Quantitative RT-PCR showed that the major adenosine receptor expressed in wild type cells was the A_{2B}R (FIG. 18a), strongly supporting intact organ culture studies that the A_{2B}R is essential for adenosine-induced cAMP production and vascular smooth muscle relaxation (FIG. 14a-c). Additionally, we showed that adenosine-mediated cAMP induction in this cell was blocked by an A_{2B}R antagonist (MRS1706) but not by an A_{2A}R antagonist (ZM241385). In addition, an A_{2A}R agonist (CGS21690C) failed to induce cAMP production in CCSM (FIG. 18b). Consistent with these results, either adenosine or NECA, a potent and broad spectrum adenosine receptor agonist, failed to induce cAMP production in CCSM of A_{2B}R deficient mice. In contrast, these agents induced cAMP in wild type CCSM. However, forskoline, an adenyl cyclase stimulator, was capable of inducing cAMP production in CCSM of both wild type and A_{2B}R-deficient mice (FIG. 18c). These results obtained from CCSM of A_{2B}R deficient mice confirm our pharmacological findings that A_{2B}R signaling is required for adenosine mediated stimulation of cAMP production in the CCSM.

Adenosine stimulates NO releasing and cGMP accumulation in CCS through A_{2B}R signaling. NO-mediated cGMP induction is a well known signaling pathway involved in penile vascular relaxation⁷⁸. However, whether adenosine is capable of inducing cGMP production in penile tissue is unknown. To test this possibility, we conducted genetic and pharmacological studies using penile organ cultures. Unexpectedly we found that cGMP production was also induced by adenosine in a dosage-dependent manner (FIG. 18d). However, the induction of cGMP production by adenosine was absent in A_{2B}R-deficient mice and was blocked by an A_{2B}R antagonist (FIG. 18d). These results indicate that adenosine-mediated cGMP production in penile tissue requires A_{2B}R activation.

Next, we explored the potential role of NO signaling in adenosine-mediated cGMP induction using penile organ cultures. We found that adenosine-mediated cGMP induction was completely blocked by L-NAME, a well known NO synthase inhibitor (FIG. 18d). This finding suggests that adenosine signaling may promote vascular smooth muscle relaxation in part by stimulating non-muscle cells to produce NO that diffuses to corpus cavernosal smooth muscle cells to activate guanylyl cyclase resulting in cGMP accumulation. To test this hypothesis, we measured cGMP levels in CCSM of wild type and A_{2B}R-deficient mice in the presence of different agents. Adenosine, NECA or forskoline had no effect on CCSM cGMP levels of either wild type or A_{2B}R deficient mice (FIG. 18e). In contrast, DEA/NO, a potent NO donor, induced cGMP production in CCSM of both wild type and A_{2B}R-deficient mice. These results suggest that adenosine-mediated cGMP, induction is via A_{2B}R-mediated NO synthesis and release from non-muscle cells resulting in cGMP induction in CCSM.

Adenosine-mediated cAMP and cGMP induction contributes to decreased phenylephrine-induced myosin light chain (MLC) phosphorylation. Pharmacological and genetic studies described above indicate that adenosine, as a potent vasodilator, has a dual role to induce both cAMP and cGMP production in the penis. To test the physiological role of adenosine-mediated induction of cAMP and cGMP on penile vascular relaxation, we examined the effect of adenosine on phenylephrine (PE)-mediated myosin light chain (MLC) phosphorylation. We found that adenosine decreased PE-mediated MLC phosphorylation in both intact penile organ culture and CCSM, showing that adenosine-mediated penile vascular relaxation is via decreased MLC phosphorylation. Interestingly, adenosine-mediated decreased MLC phosphorylation was partially inhibited by L-NAME in CCS but not in CCSM (FIG. 18f), suggesting that decreased phosphorylation of MLC by adenosine in the penis is dependent on both cGMP and cAMP production, while the adenosine-mediated cGMP production in CCSM is dependent on NO released from neighboring cells. Taken together, these findings are consistent with the view that adenosine-mediated induction of both cAMP and cGMP contributes to vascular relaxation by decreasing MLC-phosphorylation.

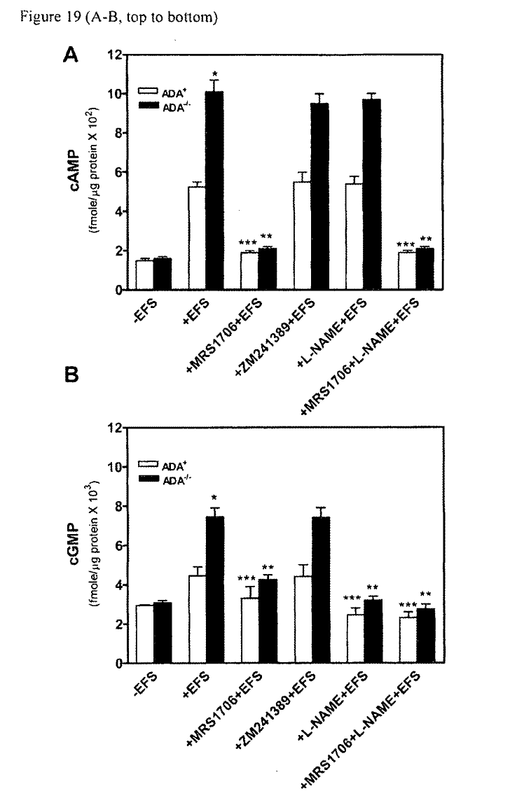

Elevated adenosine increased cAMP and cGMP production via enhanced A_{2B}R signaling in ADA deficient mice. The essential role of A_{2B}R activation in adenosine-mediated penile vascular relaxation in wild type mice led us to speculate that elevated adenosine in ADA-deficient mice may lead to enhanced adenosine signaling via A_{2B}R activation. To test this possibility, we measured levels of cAMP and cGMP in CCS of both ADA⁺ and ADA^{-/-} mice in response to EFS stimulation. We found that EFS increased cAMP and cGMP levels in both CCS of ADA⁺ and ADA^{-/-} mice (FIGS. 19a and b). However, the concentrations of cAMP and cGMP in CCS of ADA^{-/-} mice were significantly higher than CCS of ADA⁺ mice. As expected, increased cAMP levels were completely blocked by an A_{2B}R antagonist (MRS1706) but not by an A_{2A}R antagonist (ZM241489) or L-NAME in CCS of either ADA^{-/-} or ADA⁺ mice. These findings indicate that EFS-mediated cAMP induction is via A_{2B}R activation in both ADA⁺ and ADA^{-/-} mice. Similarly, we found that the increased levels of cGMP in both CCS of ADA⁺ and ADA^{-/-} mice were significantly inhibited by an A_{2B}R antagonist (MRS1706) but not by an A_{2A}R antagonist (ZM241489). L-NAME completely blocked cGMP induction in both CCS of ADA⁺ and ADA^{-/-} mice, indicating that EFS-induced cGMP induction in CCS of ADA-deficient mice is also through enhanced NO signaling via A_{2B}R activation. Taken together, these results imply that the priapism seen in ADA-deficient mice is associated with a increase in both cAMP and cGMP levels that results from adenosine mediated A_{2B}R activation.

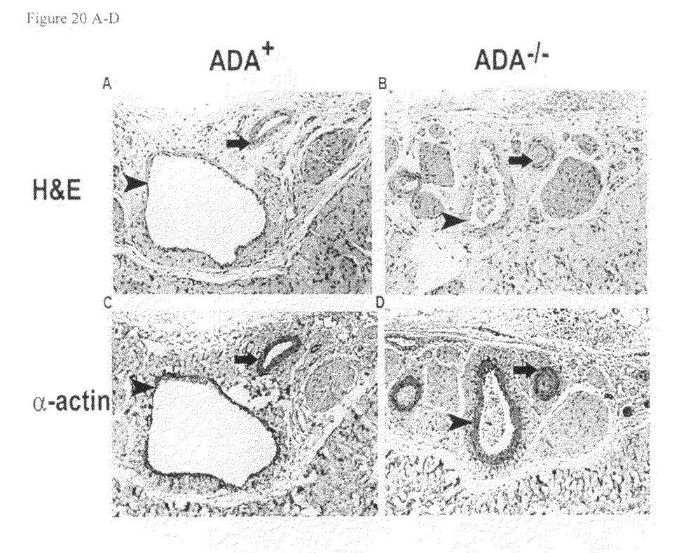

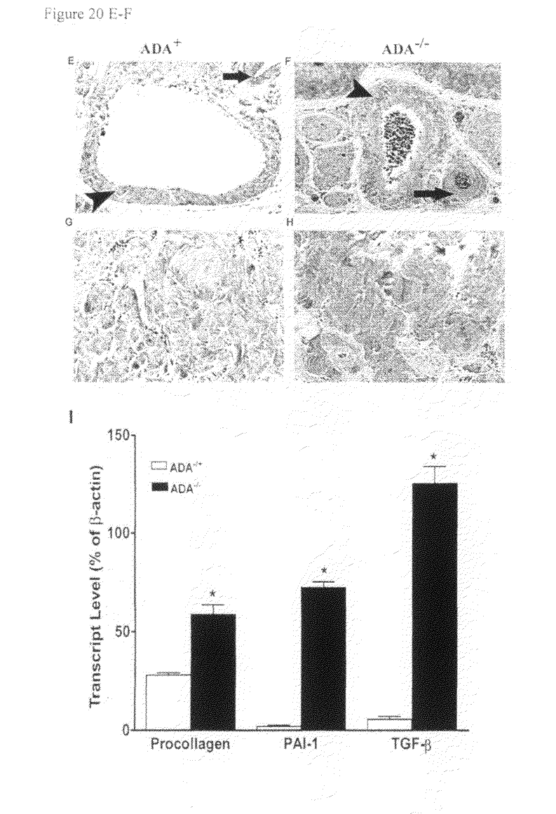

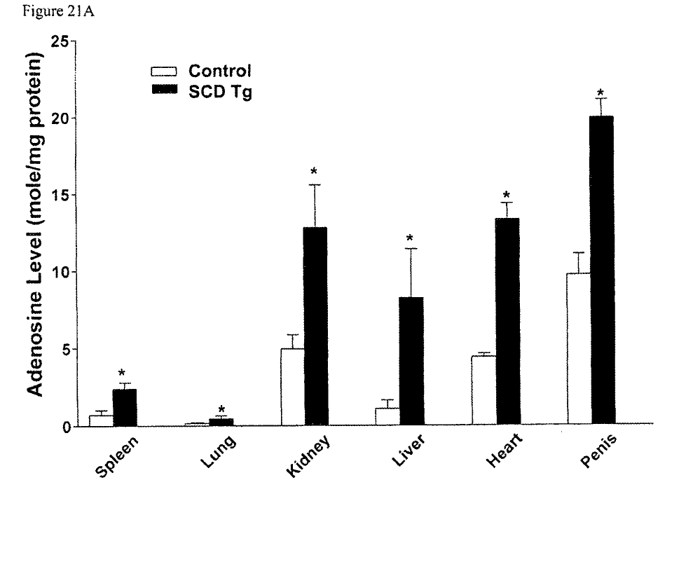

ADA-deficient mice develop penile vascular damage and fibrosis. Major complications of priapism are vascular damage and fibrosis³. Multiple studies indicate that adenosine plays an important role in vascular injury and fibrosis^{26,66-68,75,76}. ADA-deficient mice develop inflammatory injury and fibrosis in multiple organs, particularly in the lung^{26,67}. Here we also observed penile vascular damage and fibrosis in ADA-deficient mice subsequent to prolonged penile erection. H&E staining and anti-smooth muscle a-actin immunostaining demonstrated extensive endothelial damage including marked intimal thickening with smooth muscle hypertrophy and endothelial swelling (arrowheads in FIGS. 20b & 17d) in the deep dorsal vein in ADA^{-/-} mice. Parallel arteries showed muscular hypertrophy of the vascular wall (arrows in FIG. 20b & d). Extensive fibrosis with extension into the intima was also seen in the deep dorsal vein (FIGS. 20e & 17f). Due to the thickening of the intima and fibrotic changes of the vessels, the lumens of both vein and arteries in corpus spongiosum were significantly narrowed (FIG. 20a-f), leading to the more rigid and less flexible vessels in the ADA^{-/-} mice. As an end results of ischemic/hypoxic damage, significant fibrosis with sclerotic changes were also observed in corpus cavernosum accompanied with loss of cellularity compared to that from ADA⁺ mice (FIG. 20g-h). In addition, the quantitative RT-PCR showed that procollagen, plasminogen activating inhibitor-1 (PAI-1) and TGF-β gene expression, biomarkers of fibrosis, were significantly increased in the penes of ADA-deficient mice subsequent to priapism (FIG. 20i). Taken together, these results demonstrate that ADA^{-/-} mice develop significant vascular damage and subsequent tissue fibrosis.

### Example 3

Through these studies we hope to gain insight into the intracellular mechanisms by which adenosine signaling may influence penile erection and priapism. Adenosine deaminase-deficient mice accumulate supraphysiological levels of adenosine leading to priapism. Other conditions leading to elevated adenosine (possibly sickle cell disease) may also result in priapism.

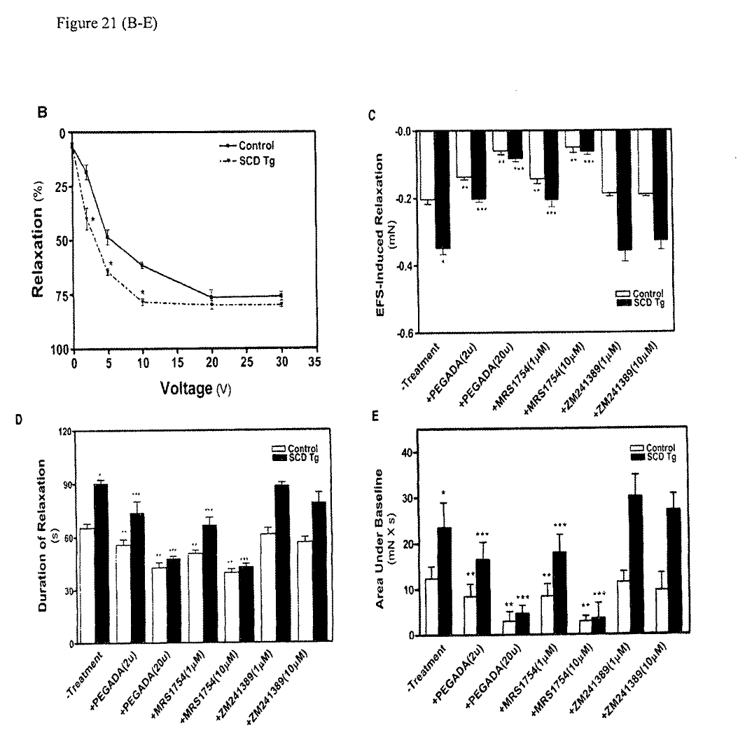

Increased adenosine contributes to priapic activity in sickle cell disease transgenic mice via A_{2B}R signaling. To assess the general significance of high adenosine in the pathophysiology of priapism, we chose to study the contributory role of adenosine in sickle cell transgenic mice because these mouse models are well accepted animal models of priapism^{19,94-97}. The adenosine levels in the penes of SCD transgenic mice were measured by HPLC and significantly higher than those of controls (FIG. 21a), suggesting that accumulated adenosine in penis may contribute to priapic activity in these mice. As with ADA^{-/-} mice, we found that CCS of SCD transgenic mice'were more sensitive to EFS stimulation than those of the controls and reached the maximum relaxation at 10V for SCD and 20V for the controls, respectively (FIG. 21b). To determine whether the increased adenosine contributed to prolonged and potent penile vascular relaxation (priapic activity) in SCD transgenic mice, we tested the effect of PEG-ADA on increased relaxation in SCD transgenic mouse CCS in response to EFS as described above. We found that treating CCS from SCD transgenic mice with PEG-ADA significantly reduced the force, duration and area under baseline when compared with untreated CCS (FIG. 21c-e). These results were very similar to the effect of PEG-ADA in priapic activity in ADA-deficient mice and suggest a general contributory role of elevated adenosine in priapism. In addition, we found that PEG-ADA treatment decreased the force, duration and area under baseline of CCS of the controls, suggesting that adenosine signaling may also involve in normal penile erection.

Next, to test whether elevated adenosine-mediated priapic activity in SCD transgenic mice is via A_{2B}R signaling as that seen in ADA-deficient mice, we measured the relaxation of CCS of both the controls and SCD transgenic mice in response to EFS in the presence or absence of A_{2B}R or A_{2A}R antagonists. We found that A_{2B}R antagonist (MRS1706) but not A_{2A}R antagonist (ZM241389) reduced the force, duration and area under baseline of the CCS of SCD transgenic mice when compared with untreated CCS (FIG. 21c-e), indicating that A_{2B}R signaling is critical for priapism in SCD transgenic mice. Similarly, A_{2B}R antagonist also decreased the force, duration and area under baseline of CCS of the controls, supporting the view that adenosine may also contribute to normal penile vascular relaxation via A_{2B}R signaling. Taken together, we show here that increased adenosine may contribute to priapic activity in SCD transgenic mice via A_{2B}R signaling.

Concluding Remarks

We used two independent mouse genetic models, ADA deficiency and SCD, as priapism models to show that elevated adenosine via A_{2B}R signaling contributes to priapism. The former model is a general model for studying the consequences of enhanced adenosine receptor signaling and the latter is a well accepted model of priapism. Thus, an unexpected priapic phenotype associated with ADA-deficient mice led us to successfully explore the unrecognized pathophysiological importance of increased adenosine signaling in priapism observed in SCD mice. Furthermore, we believe adenosine signaling in the penis may represent an important therapeutic target in the treatment of priapism.

Penile vascular tone is a key regulator of penile erection. In recent years, nitric oxide (NO) has been the major focus of studies concerning normal^{20,78} and abnormal penile vascular regulation⁴. The focus on NO was initiated by the unexpected observation by cardiologists who recognized that hypertensive patients, when treated with phosphodiesterase-5A (PDE5) inhibitors, frequently displayed persistent penile erections. These unexpected observations during clinical trials of PDE5 inhibitors drew attention to the potential role of NO in penile vascular regulation. However, studies of the role of adenosine signaling in penile vascular function are very limited. Earlier studies in several animal species, including humans³⁵, showed that intracavernous injection of adenosine resulted in tumescence and penile erection²⁹⁻³⁴. These findings suggest that adenosine may contribute to normal penile erection. Faria, et al., recently reported that corpus cavernosal tissue from erectile dysfunction patients is partially resistant to adenosine mediated relaxation due to A_{2B}R dysfunction⁷⁷. However, the role of adenosine signaling in priapism is unrecognized. One possible reason is that adenosine has a very short half life and local injection fails to accumulate at a high level for a prolonged time period. Reminiscent of the unexpected effects of PDE5 inhibitors on penile vascular relaxation found in hypertensive patients, we surprisingly discovered that increased penile adenosine concentration in ADA deficient mice induced spontaneously prolonged penile erection. This finding leads us to hypothesize that persistent elevated adenosine may contribute to priapism. This hypothesis was supported by the finding that priapic activity in SCD transgenic mouse, a well accepted priapism animal model, is also due to elevated adenosine. Thus, the unexpected experimental findings with ADA-deficient mice were confirmed and extended by the finding obtained from SCD transgenic mice, a well accepted animal model of priapism. Overall, we believe our findings with ADA deficient mice and SCD mice provide strong support for the view that elevated adenosine signaling contributes to priapism.

The elevation of adenosine in ADA-deficient mice is a direct metabolic consequence of the enzyme deficiency. However, other conditions could also lead to elevations in adenosine in the penis. Adenosine is a signaling molecule that is generated at sites of hypoxia, tissue injury and inflammation. Consistent with this, adenosine levels are usually elevated in tissues following ischemia. The most common form of priapism is the ischemic type⁹. In this regard it is noteworthy that 40% of sickle cell disease patients have episodes of priapism⁹. The priapism seen in sickle cell disease patients is usually induced by hypoxia or ischemia and is highly associated with hemolysis¹⁻². These conditions are likely to result in high local concentrations of adenosine, sufficient to induce prolonged penile erection. As expected, using the well accepted priapic mouse model, SCD transgenic mice, we show that elevated adenosine contribute to priapic activity via A_{2B}R signaling in these mice, supporting the significant pathophysiological role of adenosine signaling in priapism in general.

Among all four adenosine receptors, A_{2B}R has the lowest affinity for adenosine⁹⁸, it is likely that they are engaged under pathological conditions once adenosine is elevated. This view is consistent with the research reported here using ADA-deficient mice and SCD mice, where we have shown that pathological conditions for each mice include elevated levels of penile adenosine. Thus, the concentrations of adenosine achieved in the penes of these mutant mice are sufficient to activate the A_{2B}R. However, physiological conditions may not be necessary to achieve high concentrations of adenosine in penile tissue to activate A_{2B}R. Unexpectedly, using adenosine receptor deficient mice, as well as selective adenosine receptor antagonists, we demonstrate that the A_{2B}R is also required for adenosine mediated normal penile vascular relaxation. In addition, we found that the A_{2B}R is the major adenosine receptor expressed in the purified primary penile vascular smooth muscle cells, a key cell type involved in the regulation of penile vascular tone. Genetic deletion of the A_{2B}R completely inhibits adenosine-mediated cAMP and cGMP induction, supporting the essential role of A_{2B}R signaling in adenosine mediated penile vascular smooth muscle relaxation and erection. This finding is consistent with recent study that adenosine may induce vasodilation of corpus cavernosum via A_{2B}R in mice⁸⁰. More surprisingly, we found that adenosine concentrations were the highest and adenosine deaminase (ADA) enzyme activity was the lowest in the penis among all the tissues we measured in wild type mice (FIGS. 19a and b). On the basis of these findings along with the other data reported in this manuscript we hypothesize that such high level of adenosine may be stored in secretory vesicles of the neuron as a neurotransmitter. Adenosine is released in response to neuronal activation, allowing a locally high concentration of adenosine to be achieved and contribute to normal penile normal erection. Because of the low levels of ADA enzyme level in penis, high levels of adenosine can last for an extended period of time and may contribute to the maintenance of penile erection.

The importance of A_{2B}R signaling in penile normal erection appears to be challenged by A_{2B}R deficient mice which are not reported to have a reproductive phenotype. It is likely that multiple factors or signaling pathways may be involved in regulating normal penile function. For example, although NO signaling is widely believed to play an important role in normal penile erection, eNOS and nNOS deficient mice have no obvious reproductive problems. Thus it is possible that NO and adenosine signaling both play important roles in regulating penile vascular tone and that the two signaling systems can compensate for each other. Taken together, both animal studies, organ culture studies and cellular studies provide strong evidence that the A_{2B}R is the major receptor involved in regulation of normal and abnormal penile vascular smooth muscle relaxation and erection.

Adenosine mediated intracellular signaling pathways play an essential role in processes such as cell differentiation, proliferation, survival, apoptosis and vascular relaxation^{58,84,85}. More recent studies demonstrate that adenosine mediated-vascular relaxation may couple to NO signaling in mouse aorta⁸⁶. Consistent with these studies, we show here that adenosine is capable of inducing cAMP induction through A_{2B}R activation in penile organ culture and CCSM. In addition, we found that adenosine also induced cGMP via A_{2B}R signaling in intact penile organ culture but not in purified CCSM. These results suggest that adenosine may contribute to cavernosal smooth muscle relaxation by stimulating neighboring non-muscle cells to produce NO that diffuses to CCSM to activate guanylyl cyclase. Endothelial cells and neuron cells are likely candidates to synthesize and release NO in response to adenosine via A_{2B}R activation in the penis. More studies are needed to define the cell types that release NO in response to adenosine in penis.

Fibrosis is a common complication of priapism¹¹. A major observation in this study was that elevated adenosine in ADA-deficient mice not only induce priapic activity but also cause vascular damage and penile fibrosis, a serious complications of priapism seen in human. Thus, it is a reasonable speculation that priapic activity in ADA deficient mice may eventually progress to erectile dysfunction due to fibrosis. In addition to penile fibrosis shown here, ADA-deficient mice, similar to SCD transgenic mice, have been shown to develop fibrosis in multiple other organs²⁵⁻²⁶. It is possible that penile fibrosis results from the direct effect of elevated adenosine and/or secondary effects of adenosine-mediated prolonged penile erection. Using PEG-ADA enzyme therapy we can quickly reverse the priapic activity in ADA-deficient mice and SCD transgenic mice, suggesting therapeutic use of PEG-ADA in priapism. However, whether lowering adenosine levels can reverse aspects of penile fibrosis in ADA-deficient mice and/or SCD transgenic mice has not been addressed in this study. In this regard it is interesting to note that lowering adenosine by PEG-ADA therapy can prevent and even reverse aspects of pulmonary inflammation and fibrosis in ADA-deficient mice²⁵⁻²⁶. Thus, the utility of ADA-deficient mice is aided significantly by the ability to use ADA enzyme therapy as a convenient experimental strategy to regulate adenosine levels, and thereby control the penile abnormalities in ADA deficient mice and SCD transgenic mice. In addition, recent studies show that treatment of CVT-6883, a selective A_{2B}R antagonist in ADA-deficient mice significantly reduces adenosine-mediated pulmonary fibrosis and enlargement of alveolar airspaces⁸⁷. We have shown here that adenosine-dependent penile vascular damage and fibrosis may function through A_{2B}R signaling. Thus, antagonism of A_{2B}R-mediated responses may prove to be a beneficial therapy for both prolonged and potent vasodilation and fibrosis in priapism associated with adenosine.

So far there is no report indicating that ADA deficient children suffer from priapism because the extensive use of PEG-ADA enzyme therapy may prevent the appearance of adenosine-dependent priapism in human. In this regard it is interesting to note that numerous non-immune phenotypes were described with ADA-deficient infants prior to the generalized use of enzyme therapy in the early 1990s⁹⁹⁻¹⁰¹. During these years before the use of enzyme therapy ADA-deficient children usually died within the first two years of life and did not achieve an age allowing the issue of priapism to be addressed. Although ADA-deficiency was originally and primarily associated with a SCID phenotype⁹⁸, it is now well accepted that ADA-deficiency is a general model for studying consequences of enhanced adenosine receptor signaling. This is especially true for ADA-deficient mice were it is possible to experimentally regulate endogenous adenosine levels by varying the dose of PEG-ADA therapy. Thus, ADA deficient mice not only show immunodeficiency which is the major symptom of ADA deficient humans, but also have multiple-organ damage and malfunction, such as lung and liver²⁸. In some cases phenotypic consequences of ADA-deficiency were initially discovered in ADA-deficient mice and only subsequently recognized in ADA-deficient children¹⁰¹. Thus, we may need to pay close attention to ADA deficient males to determine whether they also present with priapism or not.

In summary, we have unexpectedly observed spontaneous prolonged penile erection in ADA-deficient mice associated with increased penile adenosine levels. We have provided extensive evidence that elevated adenosine via A_{2B}R signaling contributes to priapism in ADA-deficient mice and SCD transgenic mice. We believe our studies reported here yield significant and important new insight concerning the novel role of adenosine signaling in penile erection, priapism and penile fibrosis. It is our hope that our effort to understand the mechanisms of adenosine signaling in priapism in both ADA-deficient mice and SCD transgenic mice helps guide future studies to assess the usefulness of adenosine-based therapeutics to treat priapism and even controls the exacerbations of penile functions.

Experimental Techniques

Mice ADA-deficient (ADA^{-/-}) mice were generated and genotyped as previously described^{21,23,24}. ADA^{-/-} mice were on a mixed background of 129/sV, C57BL/6 and FVB/N strains. Control mice, designated ADA⁺, were littermates that were either wild type (+/+) or heterozygotes (+/−) for the null Ada allele as heterozygous mice do not display a phenotype. All phenotypic comparisons were performed among littermates. The four adenosine receptor deficient mice a were obtained from the following sources: A₁R-deficient mice (Jurgen Schnermann, NIDDK, NIH); A_{2A}R-deficient mice (Jiang-Fan Chen, Boston University School of Medicine); A_{2B}R-deficient mice (Michael R. Blackburn, University of Texas-Houston Medical School); and A₃R-deficient mice (Marlene Jacobson, Merck Research Laboratories). All receptor deficient mice were backcrossed at least 10 generations onto the C57Blk/6J background and were genotyped according to established protocols⁸⁸⁻⁹¹.

Sickle cell disease (SCD) transgenic mice, expressing exclusively human sickle hemoglobin, were also used. These mice were age-matched, adult male homozygotes [SS^{-/-}, consisting of a transgene and two targeted gene deletions: (Tg (Hu-miniLCR alpha 1G gamma A gamma delta beta s) Hba0//Hba0 Hbb0//Hbb00) and heterozygotes (SS^{-/+}, SS^{-/-} genotype except Hbb0//Hbb+), developed on a mixed-strain background (mouse strains FVB/N, 129, DBA/2, C57BL/6, Black Swiss)^{94,95}.

All mice were maintained and housed in accordance with the Animal Care Committee at the University of Texas Health Science Center at Houston and National Institutes of Health guidelines. Mice were housed in ventilated cages equipped with microisolator lids and maintained under strict containment protocols. No evidence of bacterial, parasitic or fungal infection was found, and sereologies on cage littermates were negative for 12 of the most common murine viruses.

ADA Enzyme Therapy Polyethlene glycol modified-ADA (PEG-ADA) was generated by the covalent modification of purified bovine ADA with activated polyethylene glycol as described^{25,26,63}. ADA^{-/-} mice were identified at birth by screening for ADA enzyme activity in the blood as described previously^{25,26,63,79}. ADA mice were maintained on ADA^{-/-} enzyme therapy since postnatal day 2. The mice were maintained with ADA enzyme therapy at least for 8 weeks when their reproductive system matured.

Quantification of penile adenosine levels Mice were anesthetized, and the penes were rapidly removed and frozen in liquid nitrogen. Adenine nucleosides were extracted from frozen penis using 0.4 N perchloric acid as described previously^{21,23}, adenosine was separated and quantified using reversed phase HPLC.

Function experiments. The tunica albuginea was cut longitudinally, starting at the most proximal point of the corpus cavernosum (CC) toward the penile shaft, and the erectile tissue was partially dissected free from the tunica. One strip of tissue (0.3×0.3×3 mm) was obtained from each corpus cavernosum. The contractility of each isolated corpus cavernosum smooth muscle cells (CCSM) strip was measured using an isometric force transducer (AD Instrument Inc., Georgia, Vt., USA). The strip was mounted in a thermostatically controlled tissue bath containing aerated PSS (mM: 119 NaCl, 4.7 KCl, 24 NaHCO 3, 1.2 KH2PO4, 2.5 CaCl2, 1.2 MgSO4, 0.023 EDTA, and 11 glucose; 5 ml volume, 95% O2 and 5% CO2, 37° C.) and stretched to a resting tension of 0.1 mN. The contractile responses of the strips were analysed by adding 10 μM phenylephrine to the bath, and force changes were recorded in response to drug application and to electrical field stimulation. The particular electrical field stimulation parameters of 0-30V, 30 HZ with a pulse width of approximately 0.5 ms, are commonly used in the mice. Available evidence indicates that these electrical field strength parameters stimulate only neurons and do not stimulate muscle, and in this way is thought to mimic normal penile erection^{73,74,93}. The changes of cAMP and cGMP contents in CC strips were subsequently measured by commercial enzyme immunoassay kit (Amersham Pharmacia) (see below).

In vitro corpus cavernosum tissue culture. Corpus cavernosum tissues were dissected as described above. The isolated corpus cavernosal strips were then immersed in standard Krebs solution (pH 7.4) bubbled with 95% O2 at 37° C. After 30-min equilibration, strips were exposed to different concentration of adenosine in the presence or absence of various adenosine receptor agonists or antagonists or L-nitroarginine methyl ester (L-NAME). 10 min later, the cavernosal strips were removed at and immediately frozen in liquid nitrogen for later analysis of cAMP and cGMP levels (see below). In a separate series of experiments, cavernosal strips were exposed to phenylephrine (10 μM) with or without treatment of adenosine or L-NAME (10 min). Subsequently cavernosal strips were removed at and immediately frozen in liquid nitrogen for phosphorylation of myosin light chain determined by western blot analysis (see below).

Isolation of primary corpus cavernosal smooth muscle cells (CCSM). Corpus cavernosum tissues were dissected as described above. Cavernosal tissue was washed in PBS and minced into 12 mm³ pieces. Segments were incubated in 5-10 ml enzyme solutions containing 0.02% collagenase A (0.272 U/mg protein, Roche, Mannheim, Germany) and 0.5% elastase (3.73 U/mg protein, Cell system, St Katharinen, Germany) in a 75 mm flask at 37° C. for 6 hours. Enzymatic digestion was terminated by adding 10 ml DMEM supplemented with 10% fetal calf serum (FCS). Afterwards, the suspension was always filtered through a 40 μM nylon mesh to separate single cells, was subsequently centrifuged at 200×g for 10 min, then resuspended and cultivated for 14 days in 75 cm² cell culture flask using 10 ml supplemented vascular smooth muscle cell growth medium as described⁷¹, including antibiotics and 10S % FCS. The purity of vascular smooth muscle was examined by smooth muscle a-action immunostaining (around 95%).

Tissue and corpus cavernosum smooth muscle cell (CCSM) cAMP and cGMP measurement. Quantitative assays for cAMP and cGMP were performed by using a commercial enzyme immunoassay kit (Amersham Pharmacia). For penile cAMP and cGMP content, frozen cavernosal tissue was homogenized in 6% trichloroacetic acid (1 ml of trichloroacetic acid per 100 mg of tissue), centrifuged, and extracted with water-saturated diethyl ether¹⁹. In studies to determine changes in cAMP and cGMP level in response to drug treatment or neurostimulation, coporal cavernosum strips excluded from previous in vitro contraction studies were snap frozen in liquid nitrogen immediately after drug treatment or nerve stimulation.

Similarly, CCSM were cultured and treated with a series of drug treatment. 10 mins later the cellular cAMP and cGMP were isolated and measured as described above.

Preparation of Protein Extracts and Western Immunoblot. Supernatants of corpus cavernosum strip homogenates and corpus cavernosum smooth muscle cells were electrophoresed and then transferred to a nitrocellulose membrane (Hybond-ECL, Amersham Pharmacia). The membranes were blocked 1 h at room temperature with blotto-Tween (5% nonfat dry milk/0.1% Tween-20) and incubated with primary myosin light chain and phospho-MLC (Calbiochem; 1:3,000) antibody. Bound antibody was detected with labeled anti-goat secondary antibody (1:20,000) (Santa Cruz Biotechnology). Enhanced chemiluminescence was performed with ECL-Plus (Amersham Pharmacia), and bands were quantified by densitometry using UN-SCAN-IT software (Silk Scientific, Orem, Utah).

Real-Time RT-PCR Analysis. Total RNA was isolated from mouse corpus cavernosum or corpus cavernosum smooth muscle cells using Trizol reagent (Invitrogen, Grand Island, N.Y.). RNase-free DNase was used to eliminate genomic DNA contamination (Invitrogen). Transcript levels were quantified using real-time quantitative RT-PCR. Adenosine receptor, procollagen, PAI-1 and TGF-β and -actin transcripts were analyzed by using Taqman probes or SYBER green on a Smart Cycler (Cepheid, Sunnyvale, Calif.), with primer sequences and conditions as previously described^{25,26}. Data were analyzed using Smart Cycler (Cepheid) analysis software. To generate a standard curve, PCR amplification was performed with template dilutions for each transcript with the assistance of the Smart Cycler program (Cepheid) to determine concentration by the cycle number where it crosses the threshold (Cτ). Final data were normalized to β-actin.

Histological analysis. Mice were anesthetized and the penes were isolated. Then the penes were pressure infused with 4% paraformaldehyde in PBS and fixed overnight at 4° C. Fixed penes were rinsed in PBS, dehydrated through graded ethanol washes, and embedded in paraffin. The histological studies including tissue sectioning and staining were performed in the Department of Pathology, Baylor College of Medicine, according to the standard protocols. Specifically, the 5 μm sections were obtained on slides. The routine hematoxylin and eosin staining and Masson's trichrome staining were performed according to the manufacture's instruction. To evaluate the expression of smooth muscle actin, the 5 μm sections of formalin-fixed, paraffin-embedded tissue were immunostained using the labeled streptavidin-biotin complex system (1:500) (Vector Labs, Burlingame, Calif.) and the primary antibody anti-SMA (1:700) (Dako Corp., Carpinteria, Calif.) after antigen retrieval using steamer.

Statistical analysis. All values are expressed as the mean±SEM. Data were analyzed for statistical significance using GraphPad Prism software. A value of P<0.05 was considered significant.

All of the methods disclosed and claimed herein can be made and executed without undue experimentation in light of the present disclosure. While the methods of this invention have been described in terms of preferred embodiments, it will be apparent to those of skill in the art that variations may be applied to the methods and in the steps or in the sequence of steps thereof described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Nolan, V. G., Wyszynski, D. F., Farrer, L. A. & Steinberg, M. H. Hemolysis-associated priapism in sickle cell disease. Blood 106, 3264-7 (2005).
2. Burnett, A. L. Therapy insight: Priapism associated with hematologic dyscrasias. Nat Clin Pract Urol 2, 449-56 (2005).
3. Burnett, A. L. Neurophysiology of erectile function: androgenic effects. J Androl 24, S2-5 (2003).
4. Lue, T. F. Erectile dysfunction. N Engl J Med 342, 1802-13 (2000).
5. Eland, I. A., van der Lei, J., Stricker, B. H. & Sturkenboom, M. J. Incidence of priapism in the general population. Urology 57, 970-2 (2001).
6. Diggs, L.a.C., RE. Pathology of sickle cell anemia. Southern Med. J. 27, 839-845 (1934).
7. Fowler, J. E., Jr., Koshy, M., Strub, M. & Chinn, S. K. Priapism associated with the sickle cell hemoglobinopathies: prevalence, natural history and sequelae. J Urol 145, 65-8 (1991).
8. Mantadakis, E., Cavender, J. D., Rogers, Z. R., Ewalt, D. H. & Buchanan, G. R. Prevalence of priapism in children and adolescents with sickle cell anemia. J Pediatr Hematol Oncol 21, 518-22 (1999).
9. Adeyoju, A. B. et al. Priapism in sickle-cell disease; incidence, risk factors and complications—an international multicentre study. BJU Int 90, 898-902 (2002).
10. Bruno, D., Wigfall, D. R., Zimmerman, S. A., Rosoff, P. M. & Wiener, J. S. Genitourinary complications of sickle cell disease. J Urol 166, 803-11 (2001).
11. Burnett, A. L. Priapism pathophysiology: clues to prevention. Int J Impot Res 15 Suppl 5, S80-5 (2003).
12. Burnett, A. L. Pathophysiology of priapism: dysregulatory erection physiology thesis. J Urol 170, 26-34 (2003).
13. Keoghane, S. R., Sullivan, M. E. & Miller, M. A. The aetiology, pathogenesis and management of priapism. BJU Int 90, 149-54 (2002).
14. Pryor, J. et al. Priapism. J Sex Med 1, 116-20 (2004).
15. Burnett, A. L. Erectile dysfunction. J Urol 175, S25-31 (2006).
16. Burnett, A. L. Erectile dysfunction: a practical approach for primary care. Geriatrics 53, 34-5, 39-40, 46-8 (1998).
17. Burnett, A. L., Allen, R. P., Tempany, C. M., Dover, G. J. & Brendler, C. B. Evaluation of erectile function in men with sickle cell disease. Urology 45, 657-63 (1995).
18. Bochinski, D. J., Dean, R. C. & Lue, T. F. Erectile dysfunction and priapism. Nat Clin Pract Urol 1, 49-53; quiz 1 p following 53 (2004).
19. Champion, H. C., Bivalacqua, T. J., Takimoto, E., Kass, D. A. & Burnett, A. L. Phosphodiesterase-5A dysregulation in penile erectile tissue is a mechanism of priapism. Proc Natl Acad Sci USA 102, 1661-6 (2005).
20. Burnett, A. L., Lowenstein, C. J., Bredt, D. S., Chang, T. S. & Snyder, S. H. Nitric oxide: a physiologic mediator of penile erection. Science 257, 401-3 (1992).
21. Blackburn, M. R., Datta, S. K., Wakamiya, M., Vartabedian, B. S. & Kellems, R. E. Metabolic and immunologic consequences of limited adenosine deaminase expression in mice. J Biol Chem 271, 15203-10 (1996).
22. Blackburn, M. R., Wakamiya, M., Caskey, C. T. & Kellems, R. E. Tissue-specific rescue suggests that placental adenosine deaminase is important for fetal development in mice. J Biol Chem 270, 23891-4 (1995).
23. Blackburn, M. R., Datta, S. K. & Kellems, R. E. Adenosine deaminase-deficient mice generated using a two-stage genetic engineering strategy exhibit a combined immunodeficiency. J Biol Chem 273, 5093-100 (1998).
24. Blackburn, M. R. & Kellems, R. E. Adenosine deaminase deficiency: metabolic basis of immune deficiency and pulmonary inflammation. Adv Immunol 86, 1-41 (2005).
25. Chunn, J. L. et al. Partially adenosine deaminase-deficient mice develop pulmonary fibrosis in association with adenosine elevations. Am J Physiol Lung Cell Mol Physiol 290, L579-87 (2006).
26. Chunn, J. L. et al. Adenosine-dependent pulmonary fibrosis in adenosine deaminase-deficient mice. J Immunol 175, 1937-46 (2005).
27. Aldrich, M. B., Blackburn, M. R., Data, S. K. & Kellems, R. E. Adenosine deaminase-deficient mice: models for the study of lymphocyte development and adenosine signaling. Adv Exp Med Biol 486, 57-63 (2000).
28. Blackburn, M. R. et al. The use of enzyme therapy to regulate the metabolic and phenotypic consequences of adenosine deaminase deficiency in mice. Differential impact on pulmonary and immunologic abnormalities. J Biol Chem 275, 32114-21 (2000).
29. Lin, C. S., Lin, G. & Lue, T. F. Cyclic nucleotide signaling in cavernous smooth muscle. J Sex Med 2, 478-91 (2005).
30. Yonezawa, A., Sakurada, S., Furukawa, K. & Kimura, Y. [Adenosine and adenosine triphosphate]. Nippon Rinsho 60 Suppl 6, 52-6 (2002).
31. Noto, T., Inoue, H., Mochida, H. & Kikkawa, K. Role of adenosine and P2 receptors in the penile tumescence in anesthetized dogs. Eur J Pharmacol 425, 51-5 (2001).
32. Filippi, S. et al. Functional adenosine receptors in human corpora cavernosa. Int J Androl 23, 210-7 (2000).
33. Shalev, M., Staerman, F., Allain, H., Lobel, B. & Saiag, B. Stimulation of P2y purinoceptors induces, via nitric oxide production, endothelium-dependent relaxation of human isolated corpus cavernosum. J Urol 161, 955-9 (1999).
34. Sharifzadeh, M., Zarrindast, M. R. & Samini, M. Effects of adenosine analogues on apomorphine-induced penile erection in rats. Gen Pharmacol 26, 1785-90 (1995).
35. Kilic, S., Salih, M., Anafarta, K., Baltaci, S. & Kosar, A. Adenosine: a new agent in the diagnosis of impotence. Int J Impot Res 6, 191-8 (1994).
36. Chiang, P. H. et al. Adenosine modulation of neurotransmission in penile erection. Br J Clin Pharmacol 38, 357-62 (1994).
37. Fredholm, B. B., AP, I. J., Jacobson, K. A., Klotz, K. N. & Linden, J. International Union of Pharmacology. XXV. Nomenclature and classification of adenosine receptors. Pharmacol Rev 53, 527-52 (2001).
38. Lad, P. M., Nielsen, T. B., Londos, C., Preston, M. S. & Rodbell, M. Independent mechanisms of adenosine activation and inhibition of the turkey erythrocyte adenylate cyclase system. J Biol Chem 255, 10841-6 (1980).
39. Cooper, D. M., Londos, C. & Rodbell, M. Adenosine receptor-mediated inhibition of rat cerebral cortical adenylate cyclase by a GTP-dependent process. Mol Pharmacol 18, 598-601 (1980).
40. Londos, C., Cooper, D. M. & Wolff, J. Subclasses of external adenosine receptors. Proc Natl Acad Sci USA 77, 2551-4 (1980).
41. Palmer, T. M. & Stiles, G. L. Identification of an A_{2A} adenosine receptor domain specifically responsible for mediating short-term desensitization. Biochemistry 36, 832-8 (1997).
42. Stiles, G. L. Adenosine Receptor Subtypes: New Insights from Cloning and Functional Studies. In Purinergic Approaches in Experimental Therapeutics. K. A. Jacobson, and M. F. Jarvis editors. New York: Wiley-Liss, Inc, 29-37 (1997).
43. Belardinelli, L., Linden, J. & Berne, R. M. The cardiac effects of adenosine. Prog Cardiovasc Dis 32, 73-97 (1989).
44. Fredholm, B. B. & Dunwiddie, T. V. How does adenosine inhibit transmitter release? Trends Pharmacol Sci 9, 130-4 (1988).
45. Churchill, P. C. Renal effects of 2-chloroadenosine and their antagonism by aminophylline in anesthetized rats. J Pharmacol Exp Ther 222, 319-23 (1982).
46. Koshiba, M., Kojima, H., Huang, S., Apasov, S. & Sitkovsky, M. V. Memory of extracellular adenosine A_{2A} purinergic receptor-mediated signaling in murine T cells. J Biol Chem 272, 25881-9 (1997).
47. Huang, S., Apasov, S., Koshiba, M. & Sitkovsky, M. Role of A_{2A} extracellular adenosine receptor-mediated signaling in adenosine-mediated inhibition of T-cell activation and expansion. Blood 90, 1600-10 (1997).
48. Rudolphi, K. A., Schubert, P., Parkinson, F. E. & Fredholm, B. B. Adenosine and brain ischemia. Cerebrovasc Brain Metab Rev 4, 346-69 (1992).
49. Schubert, P., Rudolphi, K. A., Fredholm, B. B. & Nakamura, Y. Modulation of nerve and glial function by adenosine—role in the development of ischemic damage. Int J Biochem 26, 1227-36 (1994).
50. Lasley, R. D., Rhee, J. W., Van Wylen, D. G. & Mentzer, R. M., Jr. Adenosine A₁ receptor mediated protection of the globally ischemic isolated rat heart. J Mol Cell Cardiol 22, 39-47 (1990).
51. Fishman, P., Bar-Yehuda, S., Farbstein, T., Barer, F. & Ohana, G. Adenosine acts as a chemoprotective agent by stimulating G-CSF production: a role for A₁ and A₃ adenosine receptors. J Cell Physiol 183, 393-8 (2000).
52. Forsythe, P., McGarvey, L. P., Heaney, L. G., MacMahon, J. & Ennis, M. Adenosine induces histamine release from human bronchoalveolar lavage mast cells. Clin Sci (Lond) 96, 349-55 (1999).
53. Forsythe, P. & Ennis, M. Adenosine, mast cells and asthma. Inflamm Res 48, 301-7 (1999).
54. Zhong, H., Chunn, J. L., Volmer, J. B., Fozard, J. R. & Blackburn, M. R. Adenosine-mediated mast cell degranulation in adenosine deaminase-deficient mice. J Pharmacol Exp Ther 298, 433-40 (2001).
55. Zhong, H. et al. Activation of murine lung mast cells by the adenosine A₃ receptor. J Immunol 171, 338-45 (2003).
56. Young, H. W. et al. A₃ adenosine receptor signaling contributes to airway inflammation and mucus production in adenosine deaminase-deficient mice. J Immunol 173, 1380-9 (2004).
57. Carbonaro, D. A. et al. In Vivo Transduction by Intravenous Injection of a Lentiviral Vector Expressing Human ADA into Neonatal ADA Gene Knockout Mice: A Novel Form of Enzyme Replacement Therapy for ADA Deficiency. Molecular Therapy In Press, Corrected Proof.
58. Jacobson, K. A. & Gao, Z. G. Adenosine receptors as therapeutic targets. Nat Rev Drug Discov 5, 247-64 (2006).
59. Yaar, R., Jones, M. R., Chen, J. F. & Ravid, K. Animal models for the study of adenosine receptor function. J Cell Physiol 202, 9-20. (2005).
60. Apasov, S. G., Blackburn, M. R., Kellems, R. E., Smith, P. T. & Sitkovsky, M. V. Adenosine deaminase deficiency increases thymic apoptosis and causes defective T cell receptor signaling. J Clin Invest 108, 131-41 (2001).
61. Thompson, L. F. et al. Metabolites from apoptotic thymocytes inhibit thymopoiesis in adenosine deaminase-deficient fetal thymic organ cultures. J Clin Invest 106, 1149-57 (2000).
62. Sun, C. X. et al. A protective role for the Aᵢ adenosine receptor in adenosine-dependent pulmonary injury. J Clin Invest 115, 35-43 (2005).
63. Chunn, J. L., Young, H. W., Banerjee, S. K., Colasurdo, G. N. & Blackburn, M. R. Adenosine-dependent airway inflammation and hyperresponsiveness in partially adenosine deaminase-deficient mice. J Immunol 167, 4676-85 (2001).
64. Mohsenin, A. & Blackburn, M. R. Adenosine signaling in asthma and chronic obstructive pulmonary disease. Curr Opin Pulm Med 12, 54-9 (2006).
65. Sezen, S. F. & Burnett, A. L. Intracavernosal pressure monitoring in mice: responses to electrical stimulation of the cavernous nerve and to intracavernosal drug administration. J Androl 21, 311-5 (2000).
66. Zhong, H., Belardinelli, L., Maa, T. & Zeng, D. Synergy between A_{2B} adenosine receptors and hypoxia in activating human lung fibroblasts. Am J Respir Cell Mol Biol 32, 2-8 (2005).
67. Ma, B. et al. Adenosine metabolism and murine strain-specific IL-4-induced inflammation, emphysema, and fibrosis 10.1172/JCI26372. J. Clin. Invest. 116, 1274-1283 (2006).
68. Volmer, J. B., Thompson, L. F. & Blackburn, M. R. Ecto-5′-Nucleotidase (CD73)-Mediated Adenosine Production Is Tissue Protective in a Model of Bleomycin-Induced Lung Injury. J Immunol 176, 4449-4458 (2006).
69. Okada, T. et al. Dominant localization of adenosine deaminase in leptomeninges and involvement of the enzyme in sleep. Biochem Biophys Res Commun 312, 29-34 (2003).
70. Li, H., Xu, L., Dunbar, J. C., Dhabuwala, C. B. & Sima, A. A. F. Effects of C-peptide on expression of eNOS and iNOS in human cavernosal smooth muscle cells. Urology 64, 622-627 (2004).
71. Pilatz, A. et al. Isolation of Primary Endothelial and Stromal Cell Cultures of the Corpus Cavernosum Penis for Basic Research and Tissue Engineering. European Urology 47, 710-719 (2005).
72. Pellegrino, D., Tota, B. & Randall, D. J. Adenosine/nitric oxide crosstalk in the branchial circulation of Squalus acanthias and Anguilla anguilla. Comparative Biochemistry and Physiology—Part A: Molecular & Integrative Physiology.
73. Werner, M. E., Zvara, P., Meredith, A. L., Aldrich, R. W., and Nelson, M. T. 2005. Erectile dysfunction in mice lacking the large-conductance calcium-activated potassium (BK) channel. J Physiol 567:545-556.
74. Hedlund, P., Aszodi, A., Pfeifer, A., Alm, P., Hofmann, F., Ahmad, M., Fässler, R., and Andersson, K. E. 2000. Erectile dysfunction in cyclic GMP-dependent kinase I-deficient mice, Proc Nall Acad Sci USA 97:2349-2354.
75. Chan, E. S., Montesinos, M. C., Fernandez, P., Desai, A., Delano, D. L., Yee, H., Reiss, A. B., Pillinger, M. H., Chen, IF., Schwarzschild, M. A., et al. 2006. Adenosine A (2A) receptors play a role in the pathogenesis of hepatic cirrhosis. Br J Pharmacol 148:11441155.
76. Chan, E. S., Fernandez, P., Merchant, A. A., Montesinos, M. C., Trzaska, S., Desai, A., Tung, C. F., Khoa, D. N., Pillinger, M. H., Reiss, A. B., et al. 2006. Adenosine A2A receptors in diffuse dermal fibrosis: pathogenic role in human dermal fibroblasts and in a murine model of scleroderma. Arthritis Rheum 54:2632-2642.
77. Faria, M., Magalhaes-Cardoso, T., Lafuente-de-Carvalho, J. M., and Correia-de-Sa, P. 2006. Corpus cavernosum from men with vasculogenic impotence is partially resistant to adenosine relaxation due to endothelial A (2B) receptor dysfunction. J Pharmacol Exp Ther 319:405-413.
78. Burnett, A. L. 1995. Role of nitric oxide in the physiology of erection. Biol Reprod 52:485-489.
79. Blackburn, M. R., Aldrich, M., Volmer, J. B., Chen, W., Zhong, H., Kelly, S., Hershfield, M. S., Datta, S. K., and Kellems, R. E. 2000. The use of enzyme therapy to regulate the metabolic and phenotypic consequences of adenosine deaminase deficiency in mice. Differential impact on pulmonary and immunologic abnormalities. J Biol Chem 275:32114-32121.
80. Tostes, R. C., Giachini, Cameiro, F. S., Leite, R., Inscho, E. W., and Webb, R. C. 2007. Determination of adenosine effects and adenosine receptors in murine corpus cavernosum. J Pharmacol Exp Ther.
81. Gao, Z., Chen, T., Weber, M. J., and Linden, J. 1999. A2B adenosine and P2Y2 receptors stimulate mitogen-activated protein kinase in human embryonic kidney-293 cells. crosstalk between cyclic AMP and protein kinase c pathways. J Biol Chem 274:5972-5980.
82. Linden, J., That, T., Figler, H., Jin, X., and Robeva, A. S. 1999. Characterization of human A (2B) adenosine receptors: radioligand binding, western blotting, and coupling to G (q) in human embryonic kidney 293 cells and HMC-1 mast cells. Mol Pharmacol 56:705-713.
83. Feoktistov, 1., Goldstein, A. E., and Biaggioni, I. 1999. Role of p38 mitogen-activated protein kinase and extracellular signal-regulated protein kinase kinase in adenosine A2B receptor-mediated interleukin-8 production in human mast cells. Mol Pharmacol 55:726-734.
84. Schulte, G., and Fredholm, B. B. 2003. The G (s)-coupled adenosine A (2B) receptor recruits divergent pathways to regulate ERK1/2 and p38. Exp Cell Res 290:168-176.
85. Schulte, G., and Fredholm, B. B. 2003. Signalling from adenosine receptors to mitogen-activated protein kinases. Cell Signal 15:813-827.
86. Ansari, H. R., Nadeem, A., Talukder, M. A. H., Sakhalkar, S., and Mustafa, S. J. 2007. Evidence for the involvement of nitric oxide in A2B receptor-mediated vasorelaxation of mouse aorta. Am J Physiol Heart Circ Physiol 292:H719-725.
87. Sun, Cₘₐₓ., Zhong, H., Mohsenin, A., Morschl, E., Chunn, J. L., Molina, J. G., Belardinelli, L., Zeng, D., and Blackburn, M. R. 2006. Role of A2B adenosine receptor signaling in adenosine-dependent pulmonary inflammation and injury. J Clin Invest 116:2173-2182.
88. Sun, D., Samuelson, L. C., Yang, T., Huang, Y., Paliege, A., Saunders, T., Briggs, J., and Schnermann, J. 2001. Mediation of tubuloglomerular feedback by adenosine: evidence from mice lacking adenosine 1 receptors. Proc Natl Acad Sci USA 98:9983-9988.
89. Nemeth, Z. H., Bleich, D., Csoka, B., Pacher, P., Mabley, J. G., Himer, L., Vizi, E. S., Deitch, E. A., Szabo, C., Cronstein, B. N., et al. 2007. Adenosine receptor activation ameliorates type 1 diabetes. Faseb J.
90. Chen, J. F., Huang, Z., Ma, J., Zhu, J., Moratalla, R., Standaert, D., Moskowitz, M. A., Fink, J. S., and Schwarzschild, M. A. 1999. A (2A) adenosine receptor deficiency attenuates brain injury induced by transient focal ischemia in mice. J Neurosci 19:9192-9200.
91. Salvatore, C. A., Tilley, S. L., Latour, A. M., Fletcher, D. S., Koller, B. H., and Jacobson, M. A. 2000. Disruption of the A (3) adenosine receptor gene in mice and its effect on stimulated inflammatory cells. J Biol Chem 275:4429-4434.
92. Filippi, S., Amerini, S., Maggi, M., Natali, A., and Ledda, F. 1999. Studies on the mechanisms involved in the ATP-induced relaxation in human and rabbit corpus cavernosum. J Urol 161:326-331.
93. Muneer, A., Cellek, S., Dogan, A., Kell, P. D., Ralph, D. J., and Minhas, S. 2004. Investigation of cavernosal smooth muscle dysfunction n low flow priapism using an in vitro in vitro model. 17:10-18.
94. Paszty, C., Brion, C. M., Manci, E., Witkowska, H. E., Stevens, M. E., Mohandas, N., and Rubin, E. M. 1997. Transgenic knockout mice with exclusively human sickle hemoglobin and sickle cell disease. Science 278:876-878.
95. Paszty, C. 1997. Transgenic and gene knock-out mouse models of sickle cell anemia and the thalassemias. Curr Opin Hematol 4:88-93.
96. Ryan, T. M., Ciavatta, D. J., and Townes, T. M. 1997. Knockout-transgenic mouse model of sickle cell disease. Science 278:873-876.
97. Hsu, L., Diwan, B., Ward, J. M., and Noguchi, C. T. 2006. Pathology of “Berkeley” sickle-cell mice includes gallstones and priapism 10.1182/blood-2005-11-4500. Blood 107:3414-3415.
98. Hershfield, M. S. 2005. New insights into adenosine-receptor-mediated immunosuppression and the role of adenosine in causing the immunodeficiency associated with adenosine deaminase deficiency. Eur J Immunol 35:25-30.
99. Hershfield, M. S. 1995. PEG-ADA replacement therapy for adenosine deaminase deficiency: an update after 8.5 years. Clin Immunol Immunopathol 76:S228-232.
100. Hershfield, M. S. 1995. PEG-ADA: an alternative to haploidentical bone marrow transplantation and an adjunct to gene therapy for adenosine deaminase deficiency. Hum Mutat 5:107-112.
101. Migchielsen, A. A., Breuer, M. L., van Roon, M. A., to Riele, H., Zurcher, C., Ossendorp, F., Toutain, S., Hershfield, M. S., Berns, A., and Valerio, D. 1995. Adenosine-deaminase-deficient mice die perinatally and exhibit liver-cell degeneration, atelectasis and small intestinal cell death. Nat Genet 10:279-287.

What is claimed is:

1. A method of treating priapism in a mammal, comprising:
administering to the mammal a composition comprising an effective amount of an inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal.

2. The method of claim 1, wherein the inhibitor of adenosine signaling is selected from the group consisting of adenosine deaminase (ADA), polyethylene-glycol modified adenosine deaminase (PEG-ADA), and 5′-ectonucleotidase inhibitors, and the inhibitor of adenosine signaling has at least the activity of decreasing adenosine levels in the mammal.

3. The method of claim 2, wherein the 5′-ectonucleotidase inhibitor is selected from the group consisting of ARL67156, APOPCP, and salts and esters thereof.

4. The method of claim 1, wherein the inhibitor of adenosine signaling is selected from the group consisting of theophylline, adenosine receptor A_{2B} antagonists, adenosine receptor A_{ZA} antagonists, and salts and esters thereof, and the inhibitor of adenosine signaling has at least the activity of inhibiting adenosine receptor activity in the mammal.

5. The method of claim 4, wherein the inhibitor of adenosine signaling has at least the activity of inhibiting adenosine receptor A_{2B} activity in the mammal.

6. The method of claim 5, wherein the inhibitor of adenosine signaling is selected from the group consisting of MRS1706, MRS1754, and CVT-6883.

7. The method of claim 1, wherein the inhibitor of adenosine signaling is selected from the group consisting of adenylyl cyclase inhibitors, protein kinase A inhibitors, and salts and esters thereof, and the inhibitor of adenosine signaling has at least the activity of inhibiting signaling pathways downstream of an adenosine receptor in the mammal.

8. The method of claim 7, wherein the adenylyl cyclase inhibitor is selected from the group consisting of SQ22536 and salts and esters thereof and the protein kinase A inhibitor is selected from the group consisting of H-89, PIK₁₄₋₂₂, and salts and esters thereof.

9. The method of claim 7, wherein the inhibited signaling pathway is downstream of an adenosine receptor selected from the group consisting of adenosine receptor A_{2B} and adenosine receptor A_{2A}.

10. The method of claim 1, wherein the mammal is Homo sapiens.

11. The method of claim 10, wherein the H. sapiens has sickle cell disease or priapism resulting from use of an erectile dysfunction medication.

12. The method of claim 1, wherein the mammal is a male of a non-human mammalian species.

13. The method of claim 12, wherein the mammal is selected from the group consisting of stallions, bulls, rams, dogs, cats, rabbits, rats, and mice.

14. The method of claim 1, wherein the composition has a creamy, lotion-like, or ointment-like consistency.

15. The method of claim 1, wherein administering is performed topically.

16. A method of treating erectile dysfunction in a mammal, comprising:
administering to the mammal a composition comprising an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal.

17. A method of diagnosing erectile dysfunction in a mammal, comprising:
administering to the mammal a composition comprising an effective amount of an activator of adenosine signaling and a pharmaceutically-acceptable carrier, wherein the activator of adenosine signaling has at least one activity selected from the group consisting of increasing adenosine levels in the mammal, increasing adenosine receptor activity in the mammal, and increasing activity in signaling pathways downstream of an adenosine receptor in the mammal, and
observing blood flow in the mammal's penis.
